(19) United States
(12) Patent Application Publication
(10) Pub. No.: US 2009/0111708 A1
(43) Pub. Date: Apr. 30, 2009

(54) POLYNUCLEOTIDES ASSOCIATED WITH AGE-RELATED MACULAR DEGENERATION AND METHODS FOR EVALUATING PATIENT RISK

(76) Inventors: Johanna M. Seddon, Boston, MA (US); Mark J. Daly, Arlington, MA (US)

Correspondence Address: FOLEY & LARDNER LLP, 111 HUNTINGTON AVENUE, 26TH FLOOR, BOSTON, MA, 02199-7610, US

(21) Appl. No.: 12/119,108

(22) Filed: May 12, 2008

Related U.S. Application Data
(60) Provisional application No. 60/917,439, filed on May 11, 2007.
(60) Provisional application No. 60/934,925, filed on Jul. 10, 2007.
(60) Provisional application No. 61/019,704, filed on Jan. 8, 2008.

(51) Int. Cl. C40B 30/04; C12Q 1/68; C07H 21/04; C40B 40/08; C40B 60/12; C40B 50/14

(52) U.S. Cl. 506/9; 435/6; 536/24.31; 506/17; 506/39; 506/30

(57) ABSTRACT

The present invention provides for certain polynucleotide sequences that have been correlated to AMD. These polynucleotides are useful as diagnostics, and are preferably used to fabricate an array, useful for screening patient samples. The array is used as part of a laboratory information management system, to store and process additional patient information in addition to the patient's genomic profile. As described herein, the system provides an assessment of the patient's risk for developing AMD, risk for disease progression, and the likelihood of disease prevention based on patient controllable factors.

### RELATED APPLICATIONS

This application claims the benefit of U.S. Provisional Application Ser. No. 60/917,439, filed May 11, 2007; U.S. Provisional Application Ser. No. 60/934,925, filed Jul. 10, 2007; and U.S. Provisional Application Ser. No. 61/019,704, filed Jan. 8, 2008. The contents of each of these applications is herein incorporated by reference in their entireties.

### STATEMENT OF SPONSORED RESEARCH

This invention was made with government support under EY011309 awarded by the National Institutes of Health. Additional funding was provided by the National Eye Institute (N01-EY-0-2127) and grant U54 RR020278 from the National Center for Research Resources. The government may have certain rights in the invention.

### BACKGROUND OF THE INVENTION

Age-related macular degeneration (AMD) is the most common geriatric eye disorder leading to blindness. Macular degeneration is responsible for visual handicap in what is estimated conservatively to be approximately 16 million individuals worldwide. Among the elderly, the overall prevalence is estimated between 5.7% and 30% depending on the definition of early AMD, and its differentiation from features of normal aging, a distinction that remains poorly understood.

Histopathologically, the hallmark of early neovascular AMD is accumulation of extracellular drusen and basal laminar deposit (abnormal material located between the plasma membrane and basal lamina of the retinal pigment epithelium) and basal linear deposit (material located between the basal lamina of the retinal pigment epithelium and the inner collageneous zone of Bruch's membrane). The end stage of AMD is characterized by a complete degeneration of the neurosensory retina and of the underlying retinal pigment epithelium in the macular area. Advanced stages of AMD can be subdivided into geographic atrophy and exudative AMD. Geographic atrophy is characterized by progressive atrophy of the retinal pigment epithelium. In exudative AMD the key phenomenon is the occurrence of choroidal neovascularisation (CNV). Eyes with CNV have varying degrees of reduced visual acuity, depending on location, size, type and age of the neovascular lesion. The development of choroidal neovascular membranes can be considered a late complication in the natural course of the disease possibly due to tissue disruption (Bruch's membrane) and decompensation of the underlying longstanding processes of AMD.

Many pathophysiological aspects as well as vascular and environmental risk factors are associated with a progression of the disease, but little is known about the etiology of AMD itself as well as about the underlying processes of complications like the occurrence of CNV. Family, twin, segregation, and case-control studies suggest an involvement of genetic factors in the etiology of AMD. The extent of heritability, number of genes involved, and mechanisms underlying phenotypic heterogeneity, however, are unknown. The search for genes and markers related to AMD faces challenges—onset is late in life, and there is usually only one generation available for studies. The parents of patients are often deceased, and the children are too young to manifest the disease. Generally, the heredity of late-onset diseases has been difficult to estimate because of the uncertainties of the diagnosis in previous generations and the inability to diagnose AMD among the children of an affected individual. Even in the absence of the ambiguities in the diagnosis of AMD in previous generations, the late onset of the condition itself, natural death rates, and small family sizes result in underestimation of genetic forms of AMD, and in overestimation of rates of sporadic disease. Moreover, the phenotypic variability is considerable, and it is conceivable that the currently used diagnostic entity of AMD in fact represents a spectrum of underlying conditions with various genetic and environmental factors involved.

There remains a strong need for improved methods of diagnosing or prognosticating AMD or a susceptibility to AMD in subjects, as well as for evaluating and developing new methods of treatment. It is an object of the invention to identify inherited risk factors that suggest an increased risk in developing AMD or predicting the onset of more aggressive forms of the disease.

### SUMMARY

The present invention is directed to methods and compositions that allow for improved diagnosis of AMD and susceptibility to AMD. The compositions and methods of the invention are directed to the unexpected discovery of genetic markers and causative polymorphisms in genes associated with the complement pathway. These markers and polymorphisms are useful for diagnosing AMD or a susceptibility to AMD, for use as drug targets, for identifying therapeutic agents, and for determining the efficacy of and a subject's responsiveness to a therapeutic treatment.

In one embodiment, the present invention is directed to a method for diagnosing AMD or a susceptibility to AMD, a protective phenotype for AMD, or a neutral genotype for AMD, comprising detecting the presence or absence of a particular allele at a polymorphic site associated with a complement pathway gene, wherein the allele is indicative of AMD or a susceptibility to AMD. In a particular embodiment, the polymorphic site is a single nucleotide polymorphism associated with complement factor 3, e.g., rs2230199 (SEQ ID NO:1), wherein the guanine allele is indicative of AMD or susceptibility to AMD, and wherein the cytosine allele can be detected by detecting a C3 polypeptide comprising a glycine at amino acid position 102. In a particular embodiment, the polymorphic site is selected from the group consisting of: rs1061170 (SEQ ID NO:2), wherein the cytidine allele is indicative of AMD or susceptibility to AMD; rs10490924 (SEQ ID NO:3), wherein the thymine allele is indicative of AMD or susceptibility to AMD; rs9332739 (SEQ ID NO:4), wherein the cytidine allele confers a protective effect against AMD; rs641153 (SEQ ID NO:5), wherein the thymine allele confers a protective effect against AMD; rs1410996 (SEQ ID NO:6), wherein the cytidine allele is indicative of AMD or susceptibility to AMD; and rs2230203 (SEQ ID NO:7), wherein the cytidine allele is indicative of AMD or susceptibility to AMD. In a particular embodiment, the presence or absence of a particular allele is detected by a hybridization assay. In a particular embodiment, the presence or absence of a particular allele is determined using a microarray. In a particular embodiment, the presence or absence of a particular allele is determined using an antibody.

In one embodiment, the present invention is directed to a method for identifying a subject who is at risk or protected from developing AMD, comprising: a) detecting the presence or absence of at least one at risk allele at rs2230199; b) detecting the presence or absence of at least one at risk allele or protective allele associated with complement factor H; c) detecting the presence or absence of at least one at risk allele or protective allele associated at LOC387715 in HTRA1; and d) detecting the presence or absence of at least one at risk allele or protective allele associated with complement factor B, wherein a subject is not at risk if the subject is one of about 20% of the population with a less than about 1% risk of developing AMD, and the subject is at risk if the subject is one of about 1% of the population with a greater than about 50% risk of developing AMD. In a particular embodiment, the presence or absence of a particular allele is detected by a hybridization assay. In a particular embodiment, the presence or absence of a particular allele is determined using a microarray.

In one embodiment, the present invention is directed to a purified polynucleotide comprising the polymorphic site and at least about six or more contiguous nucleotides of one or more of the sequences given as SEQ ID NOS:1-7, wherein the variant allele is present at the polymorphic site.

In one embodiment, the present invention is directed to a diagnostic array comprising one or more polynucleotide probes of the invention, e.g., probes that are complementary to a polynucleotide of the invention. In one embodiment, the invention is directed to a diagnostic system comprising: a diagnostic array of the invention, an array reader, an image processor, a database having data records and information records, a processor, and an information output; wherein the system compiles and processes patient data and outputs information relating to the statistical probability of the patient developing AMD.

In one embodiment, the present invention is directed to a method of using the diagnostic system of the invention, comprising contacting a subject sample to the diagnostic array under high stringency hybridization conditions; inputting patient information into the system; and obtaining from the system information relating to the statistical probability of the patient developing AMD.

In one embodiment, the present invention is directed to a method of making a diagnostic array of the invention comprising: applying to a substrate at a plurality particular address on the substrate a sample of the individual purified polynucleotide compositions comprising SEQ ID NOS:1-7.

In one embodiment, the present invention is directed to a method for diagnosing AMD or a susceptibility to AMD in a subject comprising combining genetic risk with behavioral risk, wherein the genetic risk is determined by detecting the presence or absence of a particular allele at a polymorphic site associated with a complement pathway gene, wherein the allele is indicative of AMD or a susceptibility to AMD. In a particular embodiment, the polymorphic site is rs2230199 (SEQ ID NO:1), wherein the guanine allele is indicative of AMD or susceptibility to AMD. In a particular embodiment, the cytosine allele is detected by detecting a C3 polypeptide comprising a glycine at amino acid position 102. In a particular embodiment, the polymorphic site is selected from the group consisting of: rs1061170 (SEQ ID NO:2), wherein the cytidine allele is indicative of AMD or susceptibility to AMD; rs10490924 (SEQ ID NO:3), wherein the thymine allele is indicative of AMD or susceptibility to AMD; rs9332739 (SEQ ID NO:4), wherein the cytidine allele confers a protective effect against AMD; rs641153 (SEQ ID NO:5), wherein the thymine allele confers a protective effect against AMD; rs1410996 (SEQ ID NO:6), wherein the cytidine allele is indicative of AMD or susceptibility to AMD; and rs2230203 (SEQ ID NO:7), wherein the cytidine allele is indicative of AMD or susceptibility to AMD. In a particular embodiment, the presence or absence of a particular allele is detected by a hybridization assay. In a particular embodiment, the presence or absence of a particular allele is determined using a microarray. In a particular embodiment, the presence or absence of a particular allele is determined using an antibody. In a particular embodiment, behavioral risk is assessed by determining if the subject exhibits a behavior or trait selected from the group consisting of: obesity, smoking, vitamin and dietary supplement intake, use of alcohol or drugs, poor diet and a sedentary lifestyle. In a particular embodiment, elevated BMI is used to determine obesity.

### BRIEF DESCRIPTION OF THE DRAWINGS

FIG. 1 is a plot showing sensitivities and specificities for a variety of risk score cutpoints and ROC curves for prediction of advanced age-related macular degeneration among younger and older age groups.

FIG. 2 are plotted histograms for advanced age-related macular degeneration risk scores for cases and controls among the original sample (above) and replication sample (below) based on all genetic variants as well as demographic and environmental variables.

FIG. 3 are sequences showing alleles at polymorphic sites: rs2230199 (SEQ ID NO:1), rs1061170 (SEQ ID NO:2), rs10490924 (SEQ ID NO:3), rs9332739 (SEQ ID NO:4), rs641153 (SEQ ID NO:5), rs1410996 (SEQ ID NO:6) and rs2230203 (SEQ ID NO:7).

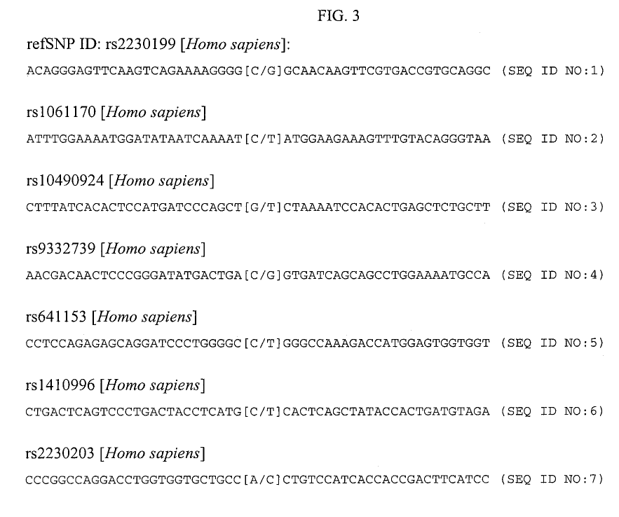

### DETAILED DESCRIPTION

The present invention is directed to the unexpected discovery that particular alleles at polymorphic sites associated with genes coding for proteins involved in the complement pathway are useful as markers for AMD and susceptibility to AMD. The compositions and methods described herein refer in particular to complement factor 3 (C3) or complement factor 5 (C5).

As used herein, “gene” is a term used to describe a genetic element that gives rise to expression products (e.g., pre-mRNA, mRNA and polypeptides). A gene includes regulatory elements and sequences that otherwise appear to have only structural features, e.g., introns and untranslated regions.

The genetic markers are particular “alleles” at “polymorphic sites” associated with particular complement factors, e.g., C3 and C5. A nucleotide position at which more than one nucleotide can be present in a population (either a natural population or a synthetic population, e.g., a library of synthetic molecules), is referred to herein as a “polymorphic site”. Where a polymorphic site is a single nucleotide in length, the site is referred to as a single nucleotide polymorphism (“SNP”). If at a particular chromosomal location, for example, one member of a population has an adenine and another member of the population has a thymine at the same genomic position, then this position is a polymorphic site, and, more specifically, the polymorphic site is a SNP. Polymorphic sites can allow for differences in sequences based on substitutions, insertions or deletions. Each version of the sequence with respect to the polymorphic site is referred to herein as an “allele” of the polymorphic site. Thus, in the previous example, the SNP allows for both an adenine allele and a thymine allele.

A genetic marker is “associated” with a genetic element or phenotypic trait, for example, if the marker is co-present with the genetic element or phenotypic trait at a frequency that is higher than would be predicted by random assortment of alleles (based on the allele frequencies of the particular population). Association also indicates physical association, e.g., proximity in the genome or presence in a haplotype block, of a marker and a genetic element.

A reference sequence is typically referred to for a particular genetic element, e.g., a gene. Alleles that differ from the reference are referred to as “variant” alleles. The reference sequence, often chosen as the most frequently occurring allele or as the allele conferring an typical phenotype, is referred to as the “wild-type” allele.

Some variant alleles can include changes that affect a polypeptide, e.g., the polypeptide encoded by a complement pathway gene. These sequence differences, when compared to a reference nucleotide sequence, can include the insertion or deletion of a single nucleotide, or of more than one nucleotide, resulting in a frame shift; the change of at least one nucleotide, resulting in a change in the encoded amino acid; the change of at least one nucleotide, resulting in the generation of a premature stop codon; the deletion of several nucleotides, resulting in a deletion of one or more amino acids encoded by the nucleotides; the insertion of one or several nucleotides, such as by unequal recombination or gene conversion, resulting in an interruption of the coding sequence of a reading frame; duplication of all or a part of a sequence; transposition; or a rearrangement of a nucleotide sequence. Alternatively, a polymorphism associated with AMD or a susceptibility to AMD can be a synonymous change in one or more nucleotides (i.e., a change that does not result in a change to a codon of a complement pathway gene). Such a polymorphism can, for example, alter splice sites, affect the stability or transport of mRNA, or otherwise affect the transcription or translation of the polypeptide. The polypeptide encoded by the reference nucleotide sequence is the “reference” polypeptide with a particular reference amino acid sequence, and polypeptides encoded by variant alleles are referred to as “variant” polypeptides with variant amino acid sequences.

Haplotypes are a combination of genetic markers, e.g., particular alleles at polymorphic sites. The haplotypes described herein are associated with AMD and/or a susceptibility to AMD. Detection of the presence or absence of the haplotypes herein, therefore is indicative of AMD, a susceptibility to AMD or a lack thereof. The haplotypes described herein are a combination of genetic markers, e.g., SNPs and microsatellites. Detecting haplotypes, therefore, can be accomplished by methods known in the art for detecting sequences at polymorphic sites.

The haplotypes and markers disclosed herein are in “linkage disequilibrium” (LD) with preferred complement pathway genes, e.g., C3 or C5, and likewise, AMD and complement-associated phenotypes. “Linkage” refers to a higher than expected statistical association of genotypes and/or phenotypes with each other. LD refers to a non-random assortment of two genetic elements. If a particular genetic element (e.g., an allele at a polymorphic site), for example, occurs in a population at a frequency of 0.25 and another occurs at a frequency of 0.25, then the predicted occurrence of a person's having both elements is 0.125, assuming a random distribution of the elements. If, however, it is discovered that the two elements occur together at a frequency higher than 0.125, then the elements are said to be in LD since they tend to be inherited together at a higher frequency than what their independent allele frequencies would predict. Roughly speaking, LD is generally correlated with the frequency of recombination events between the two elements. Allele frequencies can be determined in a population, for example, by genotyping individuals in a population and determining the occurrence of each allele in the population. For populations of diploid individuals, e.g., human populations, individuals will typically have two alleles for each genetic element (e.g., a marker or gene).

The invention is also directed to markers identified in a “haplotype block” or “LD block”. These blocks are defined either by their physical proximity to a genetic element, e.g., a complement pathway gene, or by their “genetic distance” from the element. Other blocks would be apparent to one of skill in the art as genetic regions in LD with the preferred complement pathway gene, e.g., C3 or C5. Markers and haplotypes identified in these blocks, because of their association with AMD and the complement pathway, are encompassed by the invention. One of skill in the art will appreciate regions of chromosomes that recombine infrequently and regions of chromosomes that are “hotspots”, e.g., exhibiting frequent recombination events, are descriptive of LD blocks. Regions of infrequent recombination events bounded by hotspots will form a block that will be maintained during cell division. Thus, identification of a marker associated with a phenotype, wherein the marker is contained within an LD block, identifies the block as associated with the phenotype. Any marker identified within the block can therefore be used to indicate the phenotype.

Additional markers that are in LD with the markers of the invention or haplotypes are referred to herein as “surrogate” markers. Such a surrogate is a marker for another marker or another surrogate marker. Surrogate markers are themselves markers and are indicative of the presence of another marker, which is in turn indicative of either another marker or an associated phenotype.

Several candidate genes have screened negatively for association with AMD. All of these results are reviewed in Haddad et al., which lists the relevant references. These include TIMP3 (Tissue inhibitor of metalloproteinases-3), IMPG2, the gene encoding the retinal interphotoreceptor matrix (IPM) proteoglycan IPM 200, VMD2 (the bestrophin gene), ELOVL4 (elongation of very long chain fatty acids), RDS (peripherin), EFEMP1 (EGF-containing fibulin-like extracellular matrix), BMD (bestrophin). One gene has been shown to have variations in the coding regions in patients with AMD, GPR75 (a G protein coupled receptor gene). Others have shown a possible association with the disease in at least one study—PON1 the (paraoxonase gene); SOD2 (manganese superoxide dismutase; APOE (apolipoprotein E), in which the F4 allele has been found to be associated with the disease in some studies and not associated in others; and CST3 (cystatin C), where one study has suggested an increased susceptibility for ARMD in CST3 B/B homozygotes. There are conflicting reports regarding the role of the ABCR (ABCA4) gene with regard to AMD.

### Identification of Complement Pathway Markers

Among other complement pathway members, C3 and C5 were selected as candidate genes for evaluation. Tag SNPs were selected from across C3 and C5, including SNP rs2230199 in C3, which was reported to have a p=2.8×10⁻⁵ in single marker tests available on the NIH dbGAP database in a genome-wide association of 400 AMD cases and 200 controls. Genotyping was performed as part of experiments using the Illumina GoldenGate assay and Sequenom iPLEX system as previously described. The study population consisted of 2,172 unrelated Caucasian individuals 60 years of age or older diagnosed based on ocular examination and fundus photography (1,238 cases of both dry and neovascular (wet) advanced AMD and 934 controls). This is the identical sample set described in detail previously by Maller et al., using the same phenotyping criteria, and previously established to show no inflation of case-control association statistics due to population substructure.

A single SNP in C3 (rs2230199; SEQ ID NO:1) exhibited significant association to AMD, with p<10⁻² and minor allele frequency of 0.21 in controls and 0.31 in cases (Table 2). This SNP creates a non-synonymous coding change (Arg102Gly) in the second exon of C3. No other SNPs typed in C3 showed individually statistically significant association (Table 3). In addition to testing all individual genotyped SNPs, multi-marker haplotype tests were used to evaluate association at untyped SNPs present on HapMap but no additional associations were found. Association at these SNPs and haplotypes were tested further, conditioning on the genotype at rs2230199, and no significant associations were observed (Table 3). Tests were also conducted to detect any difference in association between the neovascular and geographic atrophy forms of AMD. No statistically significant differences were observed. No SNPs in C5 exhibited significant association to AMD (Table 4).

The role of epistasis between rs2230199 and five variants was also evaluated. Two variants at CFH (1061170—SEQ ID NO:2 and 10490924—SEQ ID NO:3), two variants at the CFB/C2 locus (9332739—SEQ ID NO:4 and 641153—SEQ ID NO:5), and one at the LOC387715/HTRA1 locus (1410996—SEQ ID NO:6) were established as unequivocally associated to AMD risk in this cohort. Using logistic regression, no statistically significant interaction terms were observed between any pair of these SNPs, the two Factor B rare protective SNPs as a category or the three haplotypes formed by the two different CFH SNPs. While weak interactions cannot be excluded, this result suggests that despite targeting the same pathway, these variants largely confer risk in an independent, log-additive fashion.

Given the independent action of this new variant, rs2230199 it was added to the multi-locus risk model from Maller et al. Since the individual and combined effects of the AMD associated variants are additive, the overall proportion of population variance in risk (assuming a prevalence of late-stage AMD in this age group to be 5%) explained by this locus is roughly 2% (assuming an underlying normal distribution N(0,1) of risk across the population). For comparison, a comparable estimate of the effects of variation at CFH, LOC387715/HTRA1 and CFB are 16%, 10% and 2.5% respectively—indicating that the individual effects of these four identified genetic factors alone explain an impressive 30% of the population variation in risk for a late-onset complex disorder with known environmental covariates. Given the frequencies and penetrances of these alleles, these independent effects when combined create genuine predictive value for late-stage AMD in the population from which these cases and controls were drawn. While in this age group the prevalence of late-stage AMD is roughly 5%, variation at these four genes can identify 20% of the population that have less than 1% risk of disease, and at the opposite end identify 1% of the population with >50% risk. Indeed in this latter category, 154 cases (out of 1238) were identified compared to only 9 controls (out of 934).

HapMap Phase II reveals few proxies for rs2230199, with only 2 SNPs correlated with r²>0.4. The first, rs2230203 (SEQ ID NO:7), is a synonymous exonic polymorphism 7.6 kb downstream, correlated with r²=0.75. The other, is 5.9 kb upstream of rs2230199 outside of the gene, also correlated with r²=0.75. The small number of proxies together with the low level of linkage disequilibrium in the region suggest that the causal allele lies within a region spanning less than 14 kb.

This associated Arg102Gly variant (SEQ ID NO:1) has been established as the molecular basis of the two common allotypes of C3: C3F (fast) and C3S (slow), so named due to a difference in electrophoretic motility. The C3F variant has been previously reported as associated to other immune-mediated conditions such as IgA nephropathy and glomerular nephritis. The variant has also been reported to influence the long term success of renal transplants, where C3S homozygote recipients had much better graft survival and function when receiving a donor kidney with a C3F allotype than a matched homozygote C3S donor. More generally, deficiencies in both C3 and CFH have been associated to the immune-mediated renal damage in membranoproliferative glomerulonephritis (MPGN). and the AMD-associated Y402H variant has also been shown to be significantly associated with MPGN underscoring a deep connection in the etiology of these two disorders. The discovery of an additional association between variation in the complement system and AMD should serve to more precisely focus functional experiments and therapeutic development on the specific activity of the alternate pathway of the complement cascade.

### Diagnostic Gene Array

In one aspect, the invention comprises an array of gene fragments, particularly including those SNPs given as SEQ ID NOS:1-7, and probes for detecting the allele at the SNPs of SEQ ID NOS:1-7. Polynucleotide arrays provide a high throughput technique that can assay a large number of polynucleotide sequences in a single sample. This technology can be used, for example, as a diagnostic tool to assess the risk potential of developing AMD using the SNPs and probes of the invention. Polynucleotide arrays (for example, DNA or RNA arrays), include regions of usually different sequence polynucleotides arranged in a predetermined configuration on a substrate, at defined x and y coordinates. These regions (sometimes referenced as “features”) are positioned at respective locations (“addresses”) on the substrate. The arrays, when exposed to a sample, will exhibit an observed binding pattern. This binding pattern can be detected upon interrogating the array. For example all polynucleotide targets (for example, DNA) in the sample can be labeled with a suitable label (such as a fluorescent compound), and the fluorescence pattern on the array accurately observed following exposure to the sample. Assuming that the different sequence polynucleotides were correctly deposited in accordance with the predetermined configuration, then the observed binding pattern will be indicative of the presence and/or concentration of one or more polynucleotide components of the sample.

Arrays can be fabricated by depositing previously obtained biopolymers onto a substrate, or by in situ synthesis methods. The substrate can be any supporting material to which polynucleotide probes can be attached, including but not limited to glass, nitrocellulose, silicon, and nylon. Polynucleotides can be bound to the substrate by either covalent bonds or by non-specific interactions, such as hydrophobic interactions. The in situ fabrication methods include those described in U.S. Pat. No. 5,449,754 for synthesizing peptide arrays, and in U.S. Pat. No. 6,180,351 and WO 98/41531 and the references cited therein for synthesizing polynucleotide arrays. Further details of fabricating biopolymer arrays are described in U.S. Pat. No. 6,242,266; U.S. Pat. No. 6,232,072; U.S. Pat. No. 6,180,351; U.S. Pat. No. 6,171,797; EP No. 0 799 897; PCT No. WO 97/29212; PCT No. WO 97/27317; EP No. 0 785 280; PCT No. WO 97/02357; U.S. Pat. Nos. 5,593,839; 5,578,832; EP No. 0 728 520; U.S. Pat. No. 5,599,695; EP No. 0 721 016; U.S. Pat. No. 5,556,752; PCT No. WO 95/22058; and U.S. Pat. No. 5,631,734. Other techniques for fabricating biopolymer arrays include known light directed synthesis techniques. Commercially available polynucleotide arrays, such as Affymetrix GeneChip™, can also be used. Use of the GeneChip™, to detect gene expression is described, for example, in Lockhart et al., Nat. Biotechnol., 14:1675, 1996; Chee et al., Science, 274:610, 1996; Hacia et al., Nat. Gen., 14:441, 1996; and Kozal et al., Nat. Med., 2:753, 1996. Other types of arrays are known in the art, and are sufficient for developing an AMD diagnostic array of the present invention.

To create the arrays, single-stranded polynucleotide probes can be spotted onto a substrate in a two-dimensional matrix or array. Each single-stranded polynucleotide probe can comprise at least 6, 7, 8, 9, 10, 11, 12, 13, 14, 15, 16, 17, 18, 19, 20, 25, or 30 or more contiguous nucleotides selected from the nucleotide sequences shown in SEQ ID NO:1-7, or the complement thereof. Preferred arrays comprise at least one single-stranded polynucleotide probe comprising at least 6, 7, 8, 9, 10, 11, 12, 13, 14, 15, 16, 17, 18, 19, 20, 25, or 30 or more contiguous nucleotides selected from the nucleotide sequences shown in SEQ ID NO:1-7, or the complement thereof.

Tissue samples from a subject can be treated to form single-stranded polynucleotides, for example by heating or by chemical denaturation, as is known in the art. The single-stranded polynucleotides in the tissue sample can then be labeled and hybridized to the polynucleotide probes on the array. Detectable labels that can be used include but are not limited to radiolabels, biotinylated labels, fluorophors, and chemiluminescent labels. Double stranded polynucleotides, comprising the labeled sample polynucleotides bound to polynucleotide probes, can be detected once the unbound portion of the sample is washed away. Detection can be visual or with computer assistance. Preferably, after the array has been exposed to a sample, the array is read with a reading apparatus (such as an array “scanner”) that detects the signals (such as a fluorescence pattern) from the array features. Such a reader preferably would have a very fine resolution (for example, in the range of five to twenty microns) for a array having closely spaced features.

The signal image resulting from reading the array can then be digitally processed to evaluate which regions (pixels) of read data belong to a given feature as well as to calculate the total signal strength associated with each of the features. The foregoing steps, separately or collectively, are referred to as “feature extraction” (U.S. Pat. No. 7,206,438). Using any of the feature extraction techniques so described, detection of hybridization of a patient derived polynucleotide sample with one of the AMD markers on the array given as SEQ ID NO:1-7 identifies that subject as having or not having a genetic risk factor for AMD, as described above.

### System for Analyzing Patient Data

In another aspect, the invention provides a system for compiling and processing patient data, and presenting a risk profile for developing AMD. A computer aided medical data exchange system is preferred. The system is designed to provide high-quality medical care to a patient by facilitating the management of data available to care providers. The care providers will typically include physicians, surgeons, nurses, clinicians, various specialists, and so forth. It should be noted, however, that while general reference is made to a clinician in the present context, the care providers may also include clerical staff, insurance companies, teachers and students, and so forth. The system provides an interface, which allows the clinicians to exchange data with a data processing system. The data processing system is linked to an integrated knowledge base and a database.

The database may be software-based, and includes data access tools for drawing information from the various resources as described below, or coordinating or translating the access of such information. In general, the database will unify raw data into a useable form. Any suitable form may be employed, and multiple forms may be employed, where desired, including hypertext markup language (HTML) extended markup language (XML), Digital Imaging and Communications in Medicine (DICOM), Health Level Seven™ (HL7), and so forth. In the present context, the integrated knowledge base is considered to include any and all types of available medical data that can be processed by the data processing system and made available to the clinicians for providing the desired medical care. In general, data within the resources and knowledge base are digitized and stored to make the data available for extraction and analysis by the database and the data processing system. Even where more conventional data gathering resources are employed, the data is placed in a form that permits it to be identified and manipulated in the various types of analyses performed by the data processing system.

The integrated knowledge base is intended to include one or more repositories of medical-related data in a broad sense, as well as interfaces and translators between the repositories, and processing capabilities for carrying out desired operations on the data, including analysis, diagnosis, reporting, display and other functions. The data itself may relate to patient-specific characteristics as well as to non-patient specific information, as for classes of persons, machines, systems and so forth. Moreover, the repositories may include devoted systems for storing the data, or memory devices that are part of disparate systems, such as imaging systems. As noted above, the repositories and processing resources making up the integrated knowledge base may be expandable and may be physically resident at any number of locations, typically linked by dedicated or open network links. Furthermore, the data contained in the integrated knowledge base may include both clinical data (e.g., data relating specifically to a patient condition) and non-clinical data. Examples of preferred clinical data include patient medical histories, patient serum and cellular antioxidant levels, and the identification of past or current environmental, lifestyle and other factors that predispose a patient to develop AMD. These include but are not limited to various risk factors such as obesity, smoking, vitamin and dietary supplement intake, use of alcohol or drugs, poor diet and a sedentary lifestyle. Non-clinical data may include more general information about the patient, such as residential address, data relating to an insurance carrier, and names and addresses or phone numbers of significant or recent practitioners who have seen or cared for the patient, including primary care physicians, specialists, and so forth.

The flow of information can include a wide range of types and vehicles for information exchange. In general, the patient can interface with clinicians through conventional clinical visits, as well as remotely by telephone, electronic mail, forms, and so forth. The patient can also interact with elements of the resources via a range of patient data acquisition interfaces that can include conventional patient history forms, interfaces for imaging systems, systems for collecting and analyzing tissue samples, body fluids, and so forth. Interaction between the clinicians and the interface can take any suitable form, depending upon the nature of the interface. Thus, the clinicians can interact with the data processing system through conventional input devices such as keyboards, computer mice, touch screens, portable or remote input and reporting devices. The links between the interface, data processing system, the knowledge base, the database and the resources typically include computer data exchange interconnections, network connections, local area networks, wide area networks, dedicated networks, virtual private network, and so forth.

In general, the resources can be patient-specific or patient-related, that is, collected from direct access either physically or remotely (e.g., via computer link) from a patient. The resource data can also be population-specific so as to permit analysis of specific patient risks and conditions based upon comparisons to known population characteristics. It should be noted that the resources can generally be thought of as processes for generating data. While many of the systems and resources will themselves contain data, these resources are controllable and can be prescribed to the extent that they can be used to generate data as needed for appropriate treatment of the patient. Exemplary controllable and prescribable resources include, for example, a variety of data collection systems designed to detect physiological parameters of patients based upon sensed signals. Such electrical resources can include, for example, electroencephalography resources (EEG), electrocardiography resources (ECG), electromyography resources (EMG), electrical impedance tomography resources (EIT), nerve conduction test resources, electronystagmography resources (ENG), and combinations of such resources. Various imaging resources can be controlled and prescribed as indicated at reference numeral. A number of modalities of such resources are currently available, such as, for example, X-ray imaging systems, magnetic resonance (MR) imaging systems, computed tomography (CT) imaging systems, positron emission tomography (PET) systems, fluorography systems, sonography systems, infrared imaging systems, nuclear imaging systems, thermoacoustic systems, and so forth. Imaging systems can draw information from other imaging systems, electrical resources can interface with imaging systems for direct exchange of information (such as for timing or coordination of image data generation, and so forth).

In addition to such electrical and highly automated systems, various resources of a clinical and laboratory nature can be accessible. Such resources may include blood, urine, saliva and other fluid analysis resources, including gastrointestinal, reproductive, and cerebrospinal fluid analysis system. Such resources can further include polymerase (PCR) chain reaction analysis systems, genetic marker analysis systems, radioimmunoassay systems, chromatography and similar chemical analysis systems, receptor assay systems and combinations of such systems. Histologic resources, somewhat similarly, can be included, such as tissue analysis systems, cytology and tissue typing systems and so forth. Other histologic resources can include immunocytochemistry and histopathological analysis systems. Similarly, electron and other microscopy systems, in situ hybridization systems, and so forth can constitute the exemplary histologic resources. Pharmacokinetic resources can include such systems as therapeutic drug monitoring systems, receptor characterization and measurement systems, and so forth. Again, while such data exchange can be thought of passing through the data processing system, direct exchange between the various resources can also be implemented.

Use of the present system involves a clinician obtaining a patient sample, and evaluation of the presence of a genetic marker in that patient indicating a predisposition (or not) for AMD, such as SEQ ID NO:1-7, alone or in combination with other known risk factors. The clinician or their assistant also obtains appropriate clinical and non-clinical patient information, and inputs it into the system. The system then compiles and processes the data, and provides output information that includes a risk profile for the patient, of developing AMD.

The present invention thus provides for certain polynucleotide sequences that have been correlated to AMD. These polynucleotides are useful as diagnostics, and are preferably used to fabricate an array, useful for screening patient samples. The array, in a currently most preferred embodiment, is used as part of a laboratory information management system, to store and process additional patient information in addition to the patient's genomic profile. As described herein, the system provides an assessment of the patient's risk for developing AMD, risk for disease progression, and likelihood of disease prevention based on patient controllable factors.

### EXEMPLIFICATION

### Example 1

Discovery of genetic variants associated with AMD: Several laboratories have now identified genetic variants associated with AMD. Several of these are in the complement pathway (CFH, BF/C2). There is also an association to a region containing several tightly linked genes on chromosome 10 (LOC387715, HTRA1) although the function of those genes and variants is not fully understood. Using our databases, a previously unrecognized common, non-coding variant in CFH was identified that substantially increases the influence of this locus on AMD and strongly replicated the associations of four other published common alleles in three genes (p values ranging from about 10-12 to 10-70), including the first confirmation of the BF/C2 locus.

Complement Pathway is involved in AMD: Genetic variants and environment play a role in AMD development and pathogenesis. Therefore, it is desirable to take both into account when determining an individual's risk. To date, the Y402H variant of complement factor H (CFH) is the most replicated and studied of several variants associated with AMD, conferring an estimated 7-fold increased risk in patients with the homozygous condition. The Y402H SNP is within the CFH binding site for heparin and C-reactive protein. Binding to these sites may be altered leading to loss of function; e.g., decreased ability to bind to targets and/or interact with CRP, thereby possibly giving rise to excessive complement activation. Assays for complement fragments are becoming increasingly useful markers for early events in immunological reactions. Because the initiation of complement activation can occur on cell surfaces as well as in the fluid phase, the activation of complement may be one of the first events that can be documented. Localized processes might always not be reflected in blood.

When classical pathway activation occurs through the binding and activation of C1 to antibodies, C4 is cleaved, producing C4a and C4b. The C4a is released locally and may gain access to the circulation. It can be detected by a commercially available ELISA kits (e.g., Pharmingen OPT-EIA) in ng/ml quantities. A similar event occurs when the lectin pathway is activated through binding of mannose binding lectin (MBL) to a carbohydrate-covered bacterial surface and the mannan-binding lectin-associated serine protease (MASP) enzymes cleave C4. C4a thus serves as a marker for activation of both the classical and lectin pathways. Many charged surfaces on microbes or other particulates including aggregates of multiple classes of immunoglobulins have been shown to activate the alternative complement pathway. The first split product released in this pathway is Bb from the cleavage of factor B. Bb can be measured in plasma by a commercial ELISA kit (e.g., Quidel) in μg/ml quantities. Complement pathways can interact with one another, so measuring components of each may be important.

If activation by any of the pathways continues, C3 is the next major protein to produce measurable fragments. C3 is initially split into 2 pieces: C3a is a small fragment that has anaphylatoxin activity, interacting through a specific C3a receptor found on many cell types, and C3b is a large fragment that has the property of binding covalently to nearby surfaces or molecules through an active thioester bond. The latter is produced by a conformational change in the molecule when the C3 convertase cleaves it. This covalent attachment leads to permanent deposits of C3b (or its subsequent cleavage fragments) on surfaces in the vicinity of complement activation. These deposits and subsequent cleavage fragments interact with C3 receptors (CR1, CR2, CR3, CR4) that are found on many cell types. This leads to immune adherence and provides a transport mechanism for the clearance of immune complexes, bacteria, viruses or whatever the C3b has become attached to. C5a and C5b-9 (membrane attack complex (MAC)) are markers of the terminal activation pathway as well.

CFH dampens the alternative pathway by three actions: 1) prevents binding of factor B to C3b, 2) binds to C3bBb (the alternative pathway C3 convertase), displacing the Bb enzymatic subunit, and 3) provides cofactor activity for Factor I, which can then cleave C3b, producing the inactive form, iC3b. Some iC3b is in the fluid-phase, and is normally below 30 μg/mL in plasma, and has low variability. When elevated, it may provide an indirect indication that CFH is functioning to inactivate C3b. Inhibition of CFH with antibody reduces the cleavage of C3b to iC3b as measured by Western blot. To determine the function of CFH in inactivating C3b, it would be desirable to measure C3b and iC3b. However, C3b assays show substantial variability. Therefore, we measure C3, which reflects certain disease states, and we also analyze the ratio of iC3b/C3 as another possible indicator of AMD risk.

Factor B provides the enzymatic subunit, Bb, of the C3 convertase, contributing to the amplification loop of the alternative pathway, and formation of C5 convertase. Whereas CFH dampens the alternative pathway, properdin stabilizes C3 and C5 convertases of the alternative pathway, thus serving to promote formation of the membrane attack complex (MAC) instead of inactivation of C3b. Whereas variants of CFH increase the risk of AMD variations in the genes encoding factor B were found to reduce the risk of AMD. Both factors B and C3 have been found important in the development of laser induced choroidal neovascularization in mouse models.

In addition to genetic considerations, environmental factors play a role in AMD risk and may affect complement levels. Smoking is an independent risk factor for AMD and has been reported to activate complement and to increase factor B levels. Smokers have been reported to have reduced CFH levels. Plasma levels of CFH are reported to vary widely in the general population (110-615 μg/mL) and the measurement of CFH may not differentiate normal from variant CFH; however, more data is needed for AMD. Therefore, to determine at-risk patients, we will also measure other possible biomarkers related to recent genetic variants associated with AMD, which may also be affected by environmental factors strongly associated with increased risk of AMD. We anticipate that iC3b (or iC3b/C3) will be most elevated in non-smokers with the CFH Y402H TT genotype and with low BMI (anticipated to have stage 1), and undetectable in CC smokers with high BMI and with advanced AMD. For CC smokers with stage 1 we anticipate that factor B levels will be lower than in those with advanced AMD (with the possible caveat of patients with protective variants of factor B). Bb, a fragment of factor B produced by activation of the alternative pathway, is a reliable marker of alternative pathway activation. Once again, ratios of Bb to B are informative with respect to the activation rate and extent of the alternative pathway, and analysis of these factors in conjunction with the C3 measures provides insight into the processes ongoing in the inflammatory lesions.

Activation of the classical pathway by beta-amyloid in Alzheimer's plaques has been demonstrated by Tenner and coworkers. Given that beta-amyloid has been identified in drusen, this mechanism might provide an initiating factor for complement activation in this disease. C1 cleaves C4 in this pathway, producing C4a and C4b. Measurement of C4a and C4d (a further breakdown product of C4b) would be expected to provide additional information regarding the processes involved in the pathology. Once C3 has been cleaved by the classical pathway C3 convertase (C4bC2a) to produce C3b, the alternative pathway can take over with more efficient production of C3 fragments, C5a and C5b. C5a is the major inflammatory component of the complement cascades, but since it has an extremely short half-life, it may not be a reliable marker for a slow activation process such as that found in AMD. SC5b-9, the terminal complement complex formed by combination of non-membrane associated MAC with S protein, is a fluid phase marker of complement activation and an indirect indicator of C5 cleavage and deposition of MAC on cell or activator surfaces. It has a longer half-life than C5a and will provide more information about the extent of complement activation occurring in the AMD patients.

The sensitive tests described herein can detect low levels of complement split products that are produced only when activation occurs, and that are associated with classical/lectin, alternative or terminal pathway activation. The CH50 assay is a functional assay that relies on the sequential activation of all nine of the classical pathway proteins. It takes a fairly large reduction in any one protein to decrease the CH50 by a significant degree. In addition, CH50 reflects the classical pathway. Because most of the more studied variants, such as CFH and factor B, are involved in the alternative pathway of complement function, CH50 is not anticipated to be affected by these variants.

Genetic approach to AMD: AMD falls into the category of complex, late-onset diseases similar to type II diabetes, Alzheimer's disease, cardiovascular disease, hypertension, etc., where the genetic contributions do not necessarily manifest with straightforward Mendelian inheritance. Instead, it is presumed that these and other complex diseases are the result of complex interaction between environmental factors and susceptibility alleles of multiple genes and that these factors only cause disease when, in combination, a threshold of susceptibility is reached. Two major hypotheses are commonly explored to search for these genetic risk factors—the “common disease/common variant hypothesis” (e.g., as suggested by the association of the APOE4 allele with Alzheimer's disease) and the hypothesis that rarer, more penetrant variants at multiple genes explain the genetic component of multifactorial disease. While there is no general agreement, and limited empirical data, to suggest which hypothesis will bear more fruit in any individual disease, it seems most likely that complex diseases with involvement of many genes may quite naturally have contributions from both common and rare variation.

To detect common, low penetrance variation, an association study is the design of choice. As previously described, common variation has been conclusively determined to play a substantial role in the heritability of AMD. Previous efforts, however, have focused almost exclusively on polymorphisms that are already known to result in changes in the coding and regulatory regions of genes. A limited knowledge of the genome, limited ability to recognize many forms of potentially functional variation from sequence context alone, and lack of true understanding of causal pathways has limited the ability to apply these techniques—which were at the same time quite costly and unproven. Many of these hurdles have been overcome. In addition to the success already noted in AMD, genome-wide association approaches have resulted in validated gene findings in obesity, cardiac repolarization and type I diabetes with similar potential in genetically complex diseases.

Plasma biomarkers in the complement system are associated with AMD and AMD progression, and these associations differ according to genotype, controlling for environmental factors.

Baseline plasma levels of the complement factors were measured in patients who are genotyped and phenotyped for AMD to determine if these markers predict risk of AMD given environmental risk factors. The study population includes: 1) Discordant sibling pairs (from families and DZ twins) with one sibling grade 3b, 4, and 5 and one sibling with grade 1 (N=100 pairs, with 200 siblings), and 2) Progressors among the siblings with transition from grades 1-4 to grades 3b, 4, and 5 or grade 4 to 5 over time (total sample 620 of whom 214 have progressed). All subjects have stored plasma samples that have never been thawed, and were collected in a manner that can be used for these lab analyses. Risk factor data was available for the sample as described above, including smoking, body mass index (BMI) and serum high-sensitivity C-reactive protein (CRP) from a different aliquot of blood drawn on the same day as the proposed plasma complement assays (for the discordant pairs). Serum CRP and plasma complement factors (from aliquots drawn on the same day at baseline) are measured for subjects in the progression aspect of the study for the prospective analyses. The sibling design has been used to show that smoking increases risk, and dietary omega-3 fatty acid intake reduces risk of AMD. Complement assays: CFH, factor B, factor I, C3 and C5 levels are measured primarily with radial immunodiffusion, using polyclonal antisera specific for the components, according to the procedures followed by the Complement Laboratory at NJC. Split products C3a, iC3b, C5a and C4a, along with the terminal complement complex (SC5b-9), are measured by ELISA using kits produced by Pharmingen BD or Quidel. Ratios iC3b:C3 and C3a:C3 are also calculated. The normal ranges established in our laboratory for these components are given in Table 1.

TABLE 1

| | Normal Range | |
|---|---|---|
| Component | (mean ± 2 standard deviations) | |
| Factor H | 160-412 | μg/mL |
| Factor I | 29-58 | μg/mL |
| Factor B | 127.6-278.5 | μg/mL |
| C3 | 66-162 | mg/dL |
| C5 | 55-113 | μg/mL |
| C4 | 11-39 | mg/dL |
| C3a | 98-857 | ng/mL |
| iC3b | 0-30.9 | μg/mL |
| Bb | 0-0.83 | μg/mL |
| SC5b-9 | 0-179 | ng/mL |
| C4a | 101-745 | ng/mL |

In the clinical laboratory, anything outside of three standard deviations is considered abnormal. Given that some of the patients may have low native components (C3, FB and C4), the ratio of the levels to the split products are predicted to be more useful than absolute amounts. Comparison of the results from the disease cohorts with the controls is extremely useful for further studies in terms of identifying the appropriate biomarkers for AMD patients. All complement split products are evaluated in specimens that have been collected in EDTA tubes, processed to obtain the EDTA-plasma rapidly after blood collection, and stored frozen in liquid nitrogen freezers. Each specimen is tested for all proteins on the first thaw, since repeated freeze-thaw cycles can produce false positive results.

Methods—CFH, factor I, factor B, C5: Radial immunodiffusion is performed by preparing 1% agarose gels containing an appropriate amount of specific antibody for the component to be measured. Wells are cut in the gel and filled with a measured amount of each test serum or plasma, control serum or plasma, and a series of at least three standards with known concentration of the component measured. After incubation of the filled gels for 72 hours at 4° C., the diameter of the precipitin ring formed by combination of the antibody with its antigen (the component being tested) is measured and the area of the precipitin ring is calculated. Using the areas of the rings formed by the standards, the concentrations of the component present in the test samples are calculated by linear regression.

C3, C4: Levels of C3 and C4 are determined by Nephelometry using a Beckman-Coulter Image instrument. C3a, C4a: ELISA using OptEIA kits from Pharmingen-BD (San Diego).

iC3b, Bb, SC5b-9: these markers are measured using kits from Quidel (San Diego, Calif.). Three in-house controls are run with each set of test samples, and the specimens are all tested in duplicate.

C-reactive protein (CRP) binds to CFH at the CCP7 where the Y402H CFH polymorphism exists. Serum CRP was observed to be elevated in patients with AMD compared to controls. CRP may also increase the risk of AMD in patients carrying at least one allele of the CFH variant. CRP activates the classical pathway upon binding to its substrate, however, CRP has also been shown to reduce the magnitude of the C5b-C9 activation.

SNP Picking: A total of 8 SNPs were genotyped across C3, and 7 SNPs across C5. SNPs were picked using Tagger (found at the world wide web site, broad.mit.edu/mpg/tagger/) and HapMap data from the CEPH population (Phase II, at the world wide web site, hapmap.org). SNPs were selected with a minor allele frequency >5% with a minimum r² of 0.8. The SNPs that were selected should have been highly representative of the genetic variance within each region of interest because they were direct proxies of other SNPs in those areas, or the SNPs were part of a multimarker haplotype made up of other selected SNPs that were themselves in strong LD.

Analyses: For the case-control comparison, conditional logistic regression was used to determine the likelihood of having advanced AMD given levels of the various complement factors and CRP values within categories of genotype, while assessing and adjusting for pack year history of smoking, body mass index, and cardiovascular disease. Effect modification between complement factors vs. CRP and complement factors vs. genotype is also determined. Risk factor data is available within the existing database and analyzed. Additional analyses are also performed to assess associations between genotype and complement factors using the general linear model. For progression, similar Cox regression analyses is applied to assess whether complement levels are associated with AMD progression, controlling for genotype, smoking, BMI, CRP, etc. Interactions and effect modification are assessed to determine if complement factors are more or less related to AMD within certain genotypes, or whether these associations vary depending on smoking status, level of BMI, etc. Power for the discordant pair analyses is adequate to detect an effect size (i.e., mean difference between groups/sd)=0.40 with 80% power based on a comparison of 100 cases and 100 controls. Power is even larger for the progression study where there are 214 progressors out of 620 subjects. Regarding multiple testing, the different complement factors tend to be highly correlated and a Bonferroni type correction would be inappropriate.

During the first years of this project 1790 individuals from 370 families were enrolled in this study. DNA has been purified for genetic studies from all individuals and family history and risk factor information was also collected on all subjects. Linkage analysis has been successful in AMD. While individual studies did not provide unequivocal evidence of gene localizations, a meta-analysis of linkage studies identified regions on chromosome 1 and 10 as showing convincing evidence of linkage, along with a handful of other regions that showed suggestive evidence. These two most compelling regions were later found to harbor the strongly associated variants at the CFH and LOC387715/HTRA1 regions. Although progress has been made, it is clear that only a portion of the overall heritability has been explained by the gene variants confirmed to date. While association approaches offer extremely efficient ways of identifying common, lower penetrance contributors to disease, they may certainly miss rarer alleles contributing to disease—even those with reasonably high penetrance. To complement the ongoing gene discovery efforts, therefore, a specific strategy that is more optimized to evaluate the contribution of rarer, higher penetrance genetic variation to AMD is described.

TABLE 2
Frequency of Gly102 in cases and controls:

| Case | Control | Chi- | | | |
|---|---|---|---|---|---|
| Freq | Freq | square | P-value | OR(GR/RR)* | OR(GG/RR)* |
| 0.31 | 0.21 | 47.89 | 4.51E−12 | 1.61 | 3.26 |

*OR = Odds Ratio, with RR genotype as the referent category.

TABLE 3

C3 Association

| | | | | | | | | |
|---|---|---|---|---|---|---|---|---|
| | | | Con- | | | | | P-value |
| | | Case | trol | | Chi- | | | cond. on |
| SNP/TEST | A1 | Freq | Freq | A2 | square | P-value | OR | rs2230199 |

| | | | | | | | | |
|---|---|---|---|---|---|---|---|---|
| rs3745568 | G | 0.11 | 0.11 | T | 0.05 | 0.827 | 0.98 | 0.0772 |
| rs2047139 | C | 0.47 | 0.49 | T | 2.11 | 0.146 | 0.91 | 0.35 |
| rs2279627 | C | 0.25 | 0.25 | G | 0.00 | 0.947 | 1.00 | 0.294 |
| rs1077667 | T | 0.20 | 0.22 | C | 4.23 | 0.040 | 0.86 | 0.0519 |
| rs8106574 | T | 0.21 | 0.24 | C | 3.44 | 0.064 | 0.87 | 0.471 |
| rs344550 | C | 0.37 | 0.36 | G | 1.09 | 0.296 | 1.07 | 0.409 |
| rs2241393 | C | 0.35 | 0.38 | G | 4.18 | 0.041 | 0.88 | 0.534 |
| rs2230199 | G | 0.31 | 0.21 | C | 47.89 | 4.51E−12 | 1.66 | X |
| rs8106574, | CC | .286 | .265 | X | 2.397 | .122 | 1.11 | |
| rs344550 | | | | | | | | |
| rs2241393, | CG | .040 | .047 | X | 1.304 | .254 | 0.85 | |
| rs3745568 | | | | | | | | |
| rs2241393, | CT | .307 | .329 | X | 2.50 | .114 | 0.90 | |
| rs3745568 | | | | | | | | |

TABLE 4

C5 Association

| | | | | | | | |
|---|---|---|---|---|---|---|---|
| | | Case | Control | | Chi- | | Odds |
| SNP | A1 | Freq | Freq | A2 | square | P-value | Ratio |

| | | | | | | | |
|---|---|---|---|---|---|---|---|
| rs1930780 | C | 0.35 | 0.35 | G | 0.01 | 0.919 | 1.01 |
| rs2159776 | C | 0.47 | 0.45 | T | 1.44 | 0.230 | 1.08 |
| rs2159777 | G | 0.49 | 0.50 | T | 0.75 | 0.387 | 0.95 |
| rs4837808 | A | 0.31 | 0.28 | G | 4.52 | 0.033 | 1.16 |
| rs2038681 | C | 0.13 | 0.13 | A | 0.58 | 0.445 | 1.08 |
| rs2057470 | A | 0.31 | 0.28 | G | 5.02 | 0.025 | 1.16 |
| rs9409230 | T | 0.05 | 0.06 | A | 1.03 | 0.310 | 0.87 |
| rs2159777, rs2159776, rs1930780 | TTG | .082 | .083 | X | .015 | 0.903 | 0.986876 |
| rs2159777, rs2159776, rs1930780 | GTG | .371 | .388 | X | 1.256 | 0.263 | 0.930343 |
| rs2159777, rs1930780 | GC | .103 | .105 | X | .016 | 0.899 | 0.978765 |
| rs2159777, rs2159776 | TC | .399 | .387 | X | .561 | 0.454 | 1,051594 |
| rs2159777, rs4837808, rs2159776 | TGT | .108 | .105 | X | .101 | 0.750 | 1.032031 |
| rs2057470, rs2159776, rs2159777 | GTT | .108 | .105 | X | .101 | 0.750 | 1.032031 |
| rs2159777, rs2159776, rs2038681 | GTA | .416 | .438 | X | 2.04 | .154 | 0.913993 |
| rs2159777, rs2159776, rs2038681 | GCA | .075 | .068 | X | .914 | .339 | 1.111288 |
| rs2159777, rs4837808, rs1930780 | TAC | .192 | .186 | X | .305 | .581 | 1.039923 |
| rs2159777, rs4837808 | TA | .247 | .227 | X | 2.403 | .121 | 1,117006 |
| rs1930780, rs2159776, rs2038681 | GCA | .166 | .157 | X | .721 | .396 | 1.068735 |

### Example 2

### Prediction Model for Advanced Atrophic and Neovascular Age-Related Macular Degeneration Based on Genetic, Demographic, and Environmental Variables

Context: Six single nucleotide polymorphisms in five genes are associated with age-related macular degeneration (AMD), but their independent effects on advanced AMD have not been evaluated, controlling for environmental factors. Shown here is the evaluation of the joint effects of genetic and environmental variables and to design and assess predictive models for potential screening.

Design, Setting, and Participants: Caucasian participants in the multi-center Age-Related Eye Disease Study with advanced AMD and visual loss (n=509 cases) or no AMD (n=222 controls) were evaluated. Advanced AMD was defined as geographic atrophy, neovascular disease. Risk factors including smoking and BMI were assessed, and DNA specimens were genotyped for the six variants in five genes: CFH, LOC387715/HTRA1, CFB, C2, and C3. Unconditional logistic regression analyses were performed. Receiver operating characteristic (ROC) curves were calculated.

Outcome Measures: Prevalence of advanced dry and neovascular AMD and predictive ability of risk scores based on sensitivity and specificity to discriminate between cases and controls.

Results: CFH Y402H, CFH rs1410996, LOC387115 A69S, C2 E318D, CFB R32Q, and C3 R102H polymorphisms were each independently related to advanced AMD, controlling for demographic factors, smoking, BMI, and vitamin/mineral treatment assignment. Multivariate odds ratios (ORs) were 3.5 (95% confidence interval (CI) 1.7-7.1) for CFH Y402H, 3.7 (95% CI 1.6-8.4) for CFH rs1410996; 25.4 (95% CI 8.6-75.1) for LOC387715 A69S; 0.3 (95% CI 0.1-0.7) for C2 E318D; 0.3 (95% CI 0.1-0.5) for CFB; and 3.6 (95% CI 1.4-9.4) for C3 R102H, comparing the homozygous risk/protective genotypes to the referent genotypes. Genetic plus environmental risk scores provided C statistics ranging from 0.803 to 0.859, which were replicated in an independent sample of 452 cases and 317 controls.

Conclusions: Six genetic variants, as well as smoking and BMI are independently related to advanced AMD causing visual loss, with excellent predictive power.

As it remains unknown whether all of these genetic and environmental factors act independently or jointly and to what extent they as a group can predict the occurrence of AMD, obtaining such information is useful for screening those at high risk due to a positive family history or those who have signs of early or intermediate disease among whom some progress to advanced stages of AMD. Early detection could potentially reduce the growing societal burden due to AMD by targeting and emphasizing modifiable habits earlier in life and recommending more frequent surveillance for those highly susceptible to the disease. Treatment trials will also benefit from such information when enrolling participants.

### Methods

### Phenotypic Data

The Age-Related Eye Disease Study (AREDS) included a randomized clinical trial to assess the effect of antioxidant and mineral supplements on risk of AMD and cataract, and a longitudinal study of AMD. Based on ocular examination and AREDS reading center photographic grading of fundus photographs, Caucasian participants in this study were divided into two main groups representing the most discordant phenotypes: no AMD with either no drusen or nonextensive small drusen (n=222), or advanced AMD with visual loss (n=509). Non-Caucasians were excluded since the distribution of advanced AMD in that population differs considerably compared with Caucasians. The advanced form of AMD, groups 3 and 4 in the original AREDS classification that include non-central and central atrophy, neovascular disease, as well as visual loss, was then reclassified into the two subtypes as either non-central or central geographic atrophy (n=136) or neovascular disease (n=373), independent of visual acuity level using the Clinical Age-Related Maculopathy Grading System, to determine whether results differed between these two (advanced dry and wet) phenotypes. Another comparison was made between unilateral or bilateral advanced AMD according to the AREDS system. Demographic and risk factor data, including education, smoking history, and body mass index, were obtained at the baseline visit from questionnaires and height and weight measurements. Antioxidant status was defined as taking antioxidants (antioxidants alone or antioxidants and zinc) or no antioxidants (placebo or zinc alone) in the clinical trial. The research protocol was approved by institutional review boards and all participants signed informed consent statements.

### Genotyping

DNA samples that were drawn beginning in 1998 were obtained from the AREDS Genetic Repository. The following six common SNPs associated with AMD were evaluated: 1) Complement Factor H (CFR) Y402H (rs0161170) in exon 9 of the CFH gene on chromosome 1q31, a change 1277T>C, resulting in a substitution of histidine for tyrosine at codon 402 of the CFH protein, 2) CFH rs1410996 is an independently associated SNP variant within intron 14 of CFH, 3) LOC387715 A69S (rs10490924 in the LOC387715/HTRA1 region of chromosome 10), a non-synonymous coding SNP variant in exon 1 of LOC387715, resulting in a substitution of the amino acid serine for alanine at codon 69, 4) Complement Factor 2 or C2 E318D (rs9332739), the non-synonymous coding SNP variant in exon 7 of C2 resulting in the amino acid glutamic acid changing to aspartic acid at codon 318, 5) Complement Factor B or CFB R32Q (rs641153), the non-synonymous coding SNP variant in exon 2 of CFB resulting in the amino acid glutamine changing to arginine at codon 32, and 6) Complement Factor 3 or C3 R102H (rs2230199), the non-synonymous coding SNP variant in exon 3 of C3 resulting in the amino acid glycine to arginine at codon 102. For the genetic variant on chromosome 10 LOC387715A69S, it remains a subject of debate whether the gene HTRA1 adjacent to it may in fact be the AMD-susceptibility gene on 10q26, however, the relevant SNPs in these two genes have been reported to be nearly perfectly correlated. Thus, while the other SNP is a promising candidate variant, rs10490924 used in this study can be considered a surrogate for the causal variant that resides in this region. Genotyping was performed using primer mass extension and MALDI-TOF MS analysis by the MassEXTEND methodology of Sequenom (San Diego, Calif.).

### Statistical Analyses

Individuals with advanced AMD, as well as the separate subtypes of dry, wet and bilateral advanced AMD, were compared to the control group of Caucasian persons with no AMD, with regard to genotype and risk factor data. Multivariate unconditional logistic regression analysis was performed to evaluate the relationships between AMD and all of the genotypes plus various risk factors, controlling for age (70 or older, younger than 70), gender, and education (high school or less, more than high school), cigarette smoking (never, past, current), and BMI, which was calculated as the weight in kilograms divided by the square of the height in meters (<25, 25-29.9, and ≧30). The AREDS assignment in the randomized clinical trial was also added to the multivariate model (taking a supplement containing antioxidants or taking study supplements containing no antioxidants). Tests for multiplicative interactions between each of the genotypes versus smoking and BMI respectively, were calculated using cross product terms according to genotype and the individual risk factors. Similar analyses were performed to assess gene-gene interactions for each combination of genes. Odds ratios and 95% confidence intervals were calculated for each risk factor and within the three genotype groups. Tests for trend for the number of risk alleles for each genetic variant (0, 1, 2) were calculated. Sensitivities and specificities for a variety of risk score cut-points were evaluated to assess the optimal use of the model for individual risk prediction, e.g., sensitivities and specificities of at least 80%. The method for calculation of the AMD risk score based on all genetic, demographic and behavioral factors is explained in Table 5. The areas under the receiver operating characteristic (ROC) curves were obtained separately for the age groups 50-69 and 70+ years. An age-adjusted concordant or “C” statistic based on the ROC curves was calculated for different combinations of genes and environmental factors to assess the probability that the risk score based on the group of risk factors in that model from a random case was higher than the corresponding risk score from a random control within the same 10 year age group. To test the reproducibility of the risk prediction model, a separate replication sample consisting of 452 cases and 317 controls was obtained from the AMD study databases using the same grading system based on ocular photographs, and computed the C statistic using the risk score derived from the original sample. ROC curves were obtained for the replication sample.

### Results

The mean ages (±SD) of cases and controls were 69.1 (±5.2) and 66.8 (±4.2) respectively. Females comprised 58% of cases and 54% of controls. Table 6 displays the relationship between genotype and covariate data among controls. There were no statistically significant associations between any of the genetic variants and the demographic, behavioral, or treatment variables. There was a non-significant trend toward an association between age and the C3 variant, with a somewhat higher proportion of the younger individuals with one or two risk alleles, or the GC or GG genotypes.

Relationships between pairs of genes were also evaluated. CFH Y402H (rs1061170) and CFH (rs1410996) were significantly related (p<0.001) as a result of linkage disequilibrium between these sites, and CFB R32Q (rs641153) was weakly related to C3 R102H (rs2230199) (p=0.03) (Table 7). No other associations between pairs of genes were statistically significant. Analysis of crude AMD prevalence rates (unadjusted odds ratios (OR)) according to genotype showed a strong positive association between each of the CFH variants and AMD with prevalence OR's of 6.9 for Y402H and 11.1 for rs1410996, respectively, as well as the LOC387715 gene (OR 18.0) (p trend <0.001) (Table 8). There was also a more modest but highly significant positive relationship between the C3 variant and AMD prevalence (OR=3.1, p trend <0.001). There were inverse associations (protective effects) between the C2 and CFB variants and AMD prevalence (ORs 0.4 and 0.3, respectively, p<0.001).

Table 9 displays multivariate adjusted associations between advanced AMD and demographic and behavioral factors controlling for all genetic variants, as well as associations between AMD and genetic factors adjusting for the environmental factors. There were positive associations between the two independent CFH variants and the combined advanced AMD group (Y402H, OR=3.5, 95% CI 1.7-7.1, p trend=0.0003); CFH rs1410996 (OR=3.7, p trend=0.0003). There were positive associations between AMD and the LOC388715 A69S variant (OR=25.4, p trend <0.0001) and C3 (OR=3.6, p trend=0.001). There were protective associations between C2 (OR=0.3, p=0.003) and CFB variant (OR=0.3, p<0.0001). There were positive independent associations with age (OR=2.8, p<0.0001), current smoking (OR=3.9, p=0.001), and past smoking (OR=1.9, p=0.004). There was a protective effect of higher education (OR=0.6, p=0.01). A borderline positive association with BMI was present (OR=1.5, p=0.11) and no significant association with gender or antioxidant treatment was seen. In general, similar associations between genes and AMD were seen for all subtypes of AMD, including unilateral and bilateral advanced AMD and dry and wet types of advanced AMD, although associations varied slightly for specific types of advanced AMD.

Interactions between each genotype versus smoking (ever/never) and BMI (25+/<25), were evaluated (Table 10). No significant interactions were found between any of the genotypes and smoking or BMI, however, there was a weak non-significant trend for a smaller effect of BMI on those with genotype CFH Y402H TT and an adverse effect of BMI for those with a risk allele (the CC and CT genotypes). Within a given genotype, smoking and higher BMI increased risk of advanced AMD. For the homozygous GG risk genotype for C3, for example, the OR for advanced AMD was 3.3 (1.0-10.9) for never smokers, and increased to 9.8 (2.0-47.5) for individuals who had ever smoked, indicating that there are main effects of both smoking and C3 genotype but no interaction effect.

Interactions between pairs of genes were assessed (Table 11). There was only one borderline significant interaction found between the CFHY402H genotype and the CFH rs1410996 genotype where there was a slightly stronger effect of the CFH rs1410996 CC genotype when the CFHY402H genotype was CT rather than TT (p=0.05).

In Table 12, C statistics are presented for models with different combinations of genetic, demographic, and environmental variables. The C statistic for model 1 based on the two previously reported genes, CFH Y402H and LOC 387715 A69S, (ref) and age, gender, education, and antioxidant treatment was 0.803±0.018. There was a significant improvement in the C statistic upon adding smoking and BMI as additional risk factors in model 2 with a C statistic of 0.822±0.017 (model 1 versus 2, p=0.027). Model 3 included all six variants together with age, gender, education and antioxidant treatment and found a C statistic of 0.846±0.016, which was a significant improvement over the corresponding two gene model (model 1 vs 3, p<0.001). When smoking and BMI were added to the basic six genetic variant model 3, the C statistic increased to 0.859±0.015, and this was a significant improvement compared with the corresponding two gene model (model 2 vs. 4, p=0.001). There was a modest improvement as well with the addition of the environmental variables to the model with the six variants (model 3 vs 4, p=0.037). It should be noted that these C statistics are higher than the Framingham risk score prediction model results for coronary heart disease (CHD).

AMD risk score was tested in a separate replication sample of 452 cases and 317 controls that were not used in constructing the algorithm. The mean ages (±SD) were 76±6.6 for cases and 72±4.4 for controls, of which 49% and 53% were male, respectively. This study population was derived from other ongoing studies of genetic and epidemiologic factors described and referred to herein. This C statistic based on the replication samples as seen in Table 4 was 0.810±0.016, which indicates excellent discrimination between cases and controls. This C statistic was calculated with adjustment for age, gender, education, smoking and BMI. For this analysis, antioxidant status was assigned as “no” since participants were not taking AREDS supplements at the time of enrollment into studies and in a previous analysis no subjects were consuming high quantities of these antioxidants in their diets. The C statistic for both the original and replication samples are comparable to or exceed the C statistic for the Framingham risk score for prediction of CHD.

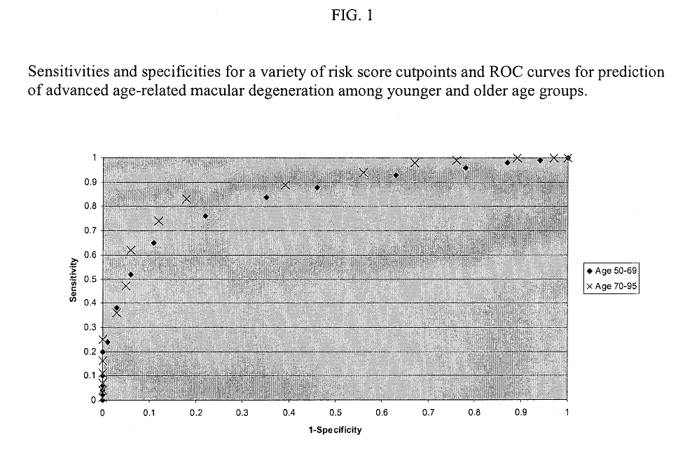

Model 4, as shown in Table 8, was considered for purposes of individual risk prediction. The sensitivity and specificity of model 4 was calculated using different cut points to denote potential screen positive criteria separately for each age group, as described in Table 5 (FIG. 1). The goal was to identify a cutpoint where both the sensitivity and specificity would be at least 80%. This was achieved for the older age group (risk score B is screen positive, <3 is screen negative), which yielded a sensitivity of 83% and specificity of 82%. Risk prediction for the younger age group was somewhat less but still good; for a cut point of screen positivity of 2.5, the sensitivity was 76% and the specificity was 78%. In general, the risk prediction was somewhat better for the older age group.

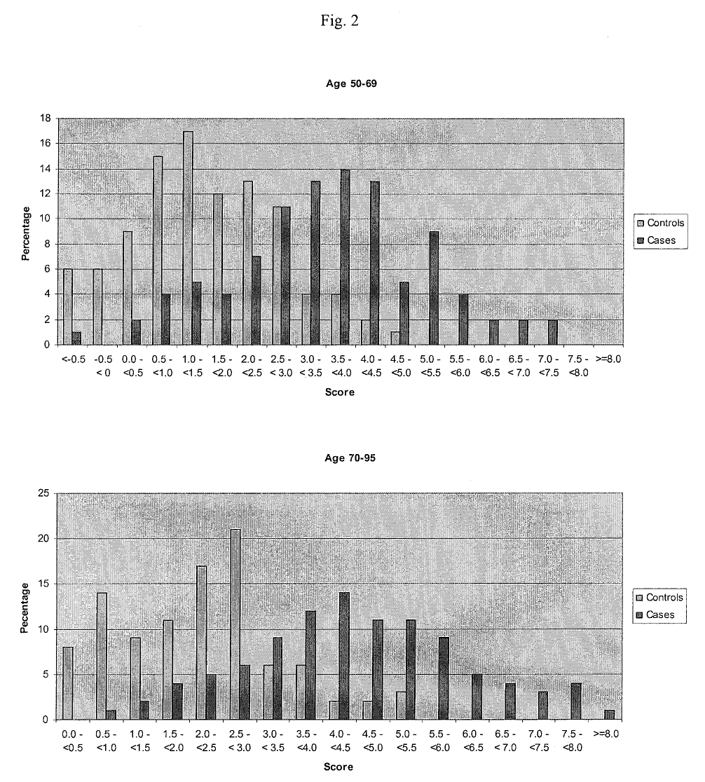

Histograms of scores for cases and controls were plotted within the two age groups (FIG. 2). Risk score distributions within a given age group appeared to be substantially different with case scores tending to be higher than controls although there was some overlap. The risk scores for the replication sample according to age and case-control status are seen at the bottom of FIG. 2 and indicate good separation between cases and controls particularly for older individuals.

### Discussion

Described herein are independent associations of six genetic variants with AMD adjusting for all of these variants in addition to demographic and behavioral factors. Discrimination between cases and controls is excellent for the overall risk score in both the original and replication samples. The predictive power of this composite of risk factors for advanced AMD, with C statistic score of 0.86 and a replication C statistic of 0.81, are comparable to or better than the Framingham risk functions for CHD in which the C statistics were 0.79 for white men and 0.83 for white women in the Framingham study cohort and somewhat lower in several replication samples. Clearly genetic factors play a major role in this disease as demonstrated by the large and consistent estimates of the effects of the genetic variants on various groups of advanced AMD, including unilateral and bilateral disease, as well as the subtypes of geographic atrophy (dry) and neovascular (wet) advanced AMD. On the other hand, modifiable factors also have an impact. Cigarette smoking increased risk for all genotypes. For example, risk of advanced AMD increased from over 3-fold for non-smokers to almost 10 fold for smokers among individuals with the same homozygous C3 risk genotype compared with non-smokers with the non-risk genotype. Higher BMI also contributed to the risk profile for all genotypes.

These analyses expand and refine observations (Example 1) in important and meaningful ways by adding a new genetic variant, incorporating demographic and behavioral factors, calculating C statistics for advanced AMD based on models with different combinations of genetic and environmental variables, and evaluating the ability of the resultant risk scores to discriminate between individuals with and without advanced AMD.

Unique features of this study include the evaluation of predictive power based on a large, well-characterized population of Caucasian patients with or without advanced AMD from various geographic regions around the US. Further strengths include the standardized collection of risk factor information, direct measurements of height and weight, and classification of maculopathy by standardized ophthalmologic examinations and grading of fundus photographs. Misclassification was unlikely since grades were assigned without knowledge of risk factors or genotype. Controls were performed for known AMD risk factors, including age, education, BMI, smoking, and treatment assignment in assessing the relationship between genetic variants and advanced AMD. Both the environmental and genetic risk factors were independently associated with AMD, when considered simultaneously. Although this is a selected population, subjects likely represent the typical patient with advanced AMD, and the overall population is similar to others in this age range in terms of smoking and prevalence of obesity, as well as the distribution of the genotypes. This large and well-characterized population provided a unique opportunity to evaluate gene-environment associations and interactions. Furthermore, the biological effects of the genetic variants do not appear to differ in major ways among various Caucasian populations with AMD.

Although it would be desirable to assess these relationships with incident AMD it is unlikely that many individuals without AMD in this elderly age group would progress to advanced disease during the remainder of their lifetime. Thus the potential for misclassification of controls who might ultimately become cases is likely to be small.

Knowledge of drusen characteristics among those with early and intermediate disease is also related to progression to advanced AMD, but this study is focused on a different subject: the discriminatory ability of genetic and non-genetic factors in predicting status as a case with advanced AMD or a control without signs of AMD. Furthermore, among individuals with high risk drusen or pigment abnormalities, two of the six genetic variants predict progression to advanced disease independent of their fundus appearance.

These analyses and results indicate the potential for individual risk prediction for AMD. In calculating the risk score, for example, one could estimate “points” from the regression coefficients (Table 5) for smoking (1.3), higher BMI (0.4), and the various genetic variants (ranging from −1.3 to +3.2) to obtain an overall risk for an individual to develop advanced AMD. This could be refined as new genetic and other risk predictors are established. Advantages of knowing such a risk score include the possibility for more targeted education and counseling about known modifiable factors. Screening would identify high risk people who would be encouraged to follow a healthy lifestyle by not smoking, eating vegetables and fish, maintaining a normal weight and getting exercise, and taking AREDS type antioxidant and mineral supplements for those with signs of AMD. All of these factors are known to influence the inflammatory and immune pathways that are involved in the pathogenesis of AMD. Targeting high risk individuals could also lead to heightened awareness and more frequent surveillance and clinical examinations, as well as identification of high risk individuals for inclusion in clinical trials of new therapies.

TABLE 5
Calculation of AMD Risk Score.
The risk score was calculated from the following formula:
$S = {\sum }_{i = 1}^{18} {β}_{i} {X}_{i} where {β}_{i} and {X}_{i} are given as follows \text{:}$

| | | Regres- | | | | | |
|---|---|---|---|---|---|---|---|
| | | sion | | | | | |
| | Variable | Coeff | | Control | | Case | |
| i | Name (Xᵢ) | (βᵢ) | Code | (X₀) | βᵢXᵢ | (Xᵢ) | βᵢXᵢ |
| 1 | Age 70+ | 1.0130 | | 0 | 0 | 0 | 0 |
| 2 | Gender | −0.1053 | 1 = | 1 | −0.11 | 1 | −0.11 |
| | | | m/0 = f | | | | |
| 3 | Education | −0.5845 | 1 = some | 1 | −0.58 | 1 | −0.58 |
| | | | college/ | | | | |
| | | | 0 = high | | | | |
| | | | school or | | | | |
| | | | less | | | | |
| 4 | Antioxidant | 0.2404 | 1 = yes/ | 0 | 0 | 1 | 0.24 |
| | Use | | 0 = no | | | | |
| 5 | BMI 25-29 | 0.0871 | 1 = yes/ | 1 | 0.09 | 1 | 0.09 |
| | | | 0 = no | | | | |
| 6 | BMI 30+ | 0.4370 | 1 = yes/ | 0 | 0 | 0 | 0 |
| | | | 0 = no | | | | |
| 7 | Current | 1.3555 | 1 = yes/ | 0 | 0 | 0 | 0 |
| | Smoking | | 0 = no | | | | |
| 8 | Past | 0.6247 | 1 = yes/ | 1 | 0.62 | 1 | 0.62 |
| | Smoking | | 0 = no | | | | |
| 9 | CFH:rs1061 | 0.6002 | 1 = yes/ | 0 | 0 | 1 | 0.60 |
| | 170 | | 0 = no | | | | |
| | (Y402H) CT | | | | | | |
| 10 | CFH:rs1061 | 1.2582 | 1 = yes/ | 0 | 0 | 0 | 0 |
| | 170 | | 0 = no | | | | |
| | (Y402H) CC | | | | | | |
| 11 | LOC387715: | 1.1238 | 1 = yes/ | 0 | 0 | 0 | 0 |
| | rs10490924 | | 0 = no | | | | |
| | (A69S) GT | | | | | | |
| 12 | LOC387715: | 3.2343 | 1 = yes/ | 0 | 0 | 1 | 3.23 |
| | rs10490924 | | 0 = no | | | | |
| | (A69S) TT | | | | | | |
| 13 | C3:rs2230199 | 0.4879 | 1 = yes/ | 0 | 0 | 0 | 0 |
| | (R102H) CG | | 0 = no | | | | |
| 14 | C3:rs2230199 | 1.2898 | 1 = yes/ | 0 | 0 | 0 | 0 |
| | (R102H) GG | | 0 = no | | | | |
| 15 | CFB:rs641153 | −1.3453 | 1 = yes/ | 1 | −1.35 | 0 | 0 |
| | (R32Q) CT | | 0 = no | | | | |
| | or TT | | | | | | |
| 16 | C2:rs9332739 | −1.1830 | 1 = yes/ | 0 | 0 | 0 | 0 |
| | (E318D) CT | | 0 = no | | | | |
| | or CC | | | | | | |
| 17 | CFH:rs1410 | 0.4989 | 1 = yes/ | 0 | 0 | 0 | 0 |
| | 996 CT | | 0 = no | | | | |
| 18 | CFH:rs1410 | 1.3004 | 1 = yes/ | 0 | 0 | 1 | 1.30 |
| | 996 CC | | 0 = no | | | | |
| | Risk Score | | | | −1.32 | | 5.4 |

TABLE 6A
Genotype-Phenotype Associations Among Controls
GENOTYPE

| | CFH: rs1061170(Y402H) | | | | | | | LOC387715: rs10490924(A69S) | | | | | | |
|---|---|---|---|---|---|---|---|---|---|---|---|---|---|---|
| | TT | | CT | | CC | | | GG | | GT | | TT | | |
| | N | % | N | % | N | % | | N | % | N | % | N | % | |
| Variable | | | | | | | | | | | | | | |
| Baseline Age ≦70 | 64 | 70.3 | 69 | 67.6 | 23 | 79.3 | | 104 | 68.9 | 48 | 71.6 | 4 | 100 | |
| 70+ | 27 | 29.7 | 33 | 32.4 | 6 | 20.7 | | 47 | 31.1 | 19 | 28.4 | 0 | | |
| p (trend) | | | | | | | 0.58 | | | | | | | 0.34 |
| Gender | | | | | | | | | | | | | | |
| Male | 42 | 46.2 | 42 | 41.2 | 17 | 58.6 | | 66 | 43.7 | 35 | 52.2 | 0 | | |
| Female | 49 | 53.8 | 60 | 58.8 | 12 | 41.4 | | 85 | 56.3 | 32 | 47.8 | 4 | 100 | |
| p (trend) | | | | | | | 0.53 | | | | | | | 0.82 |
| Education | | | | | | | | | | | | | | |
| High School or | 24 | 26.4 | 30 | 29.4 | 10 | 34.5 | | 44 | 29.1 | 19 | 28.4 | 1 | 25.0 | |
| Less | | | | | | | | | | | | | | |
| College or | 67 | 73.6 | 72 | 70.6 | 19 | 65.5 | | 107 | 70.9 | 48 | 71.6 | 3 | 75.0 | |
| More | | | | | | | | | | | | | | |
| p (trend) | | | | | | | 0.40 | | | | | | | 0.86 |
| Smoking Baseline | | | | | | | | | | | | | | |
| Never | 43 | 47.3 | 60 | 58.8 | 11 | 37.9 | | 81 | 53.6 | 30 | 44.8 | 3 | 75.0 | |
| Past | 42 | 46.2 | 38 | 37.3 | 17 | 58.6 | | 62 | 41.1 | 34 | 50.7 | 1 | 25.0 | |
| Current | 6 | 6.6 | 4 | 3.9 | 1 | 3.4 | | 8 | 5.3 | 3 | 4.5 | 0 | | |
| p (trend) | | | | | | | 0.77 | | | | | | | 0.69 |
| BMI Baseline | | | | | | | | | | | | | | |
| <25 | 28 | 30.8 | 37 | 36.3 | 9 | 31 | | 51 | 33.8 | 22 | 32.8 | 1 | 33.3 | |
| 25-29 | 34 | 37.4 | 47 | 46.1 | 15 | 51.7 | | 65 | 43.0 | 30 | 44.8 | 1 | 33.3 | |
| ≧30 | 29 | 31.9 | 18 | 17.6 | 5 | 17.2 | | 35 | 23.2 | 15 | 22.4 | 1 | 33.3 | |
| p (trend) | | | | | | | 0.14 | | | | | | | 0.67 |
| Antioxidants | | | | | | | | | | | | | | |
| Yes | 37 | 40.7 | 50 | 49.0 | 11 | 37.9 | | 68 | 45.0 | 28 | 41.8 | 2 | 50.0 | |
| No | 54 | 59.3 | 52 | 51.0 | 18 | 62.1 | | 83 | 55.0 | 39 | 58.2 | 2 | 50.0 | |
| p (trend) | | | | | | | 0.79 | | | | | | | 0.77 |

TABLE 6B
Genotype-Phenotype Associations Among Controls
Genotype

| | CFH: rs1410996 | | | | | | | C2: rs9332739(E318D) | | | | |
|---|---|---|---|---|---|---|---|---|---|---|---|---|
| | TT | | CT | | CC | | | TT | | CT or CC | | |
| | N | % | N | % | N | % | | N | % | N | % | |
| Variable | | | | | | | | | | | | |
| Baseline Age ≦70 | 30 | 75.0 | 78 | 66.7 | 48 | 73.8 | | 140 | 71.1 | 16 | 64.0 | |
| 70+ | 10 | 25.0 | 39 | 33.3 | 17 | 26.2 | | 57 | 28.9 | 9 | 36.0 | |
| p (trend) | | | | | | | 0.93 | | | | | 0.47 |
| Gender | | | | | | | | | | | | |
| Male | 17 | 42.5 | 52 | 44.4 | 32 | 49.2 | | 93 | 47.2 | 8 | 32.0 | |
| Female | 23 | 57.5 | 65 | 55.6 | 33 | 50.8 | | 104 | 52.8 | 17 | 68.0 | |
| p (trend) | | | | | | | 0.47 | | | | | 0.15 |
| Education | | | | | | | | | | | | |
| High School) | 11 | 27.5 | 28 | 23.9 | 25 | 38.5 | | 58 | 29.4 | 6 | 24.0 | |
| College+ | 29 | 72.5 | 89 | 76.1 | 40 | 61.5 | | 139 | 70.6 | 19 | 76.0 | |
| p (trend) | | | | | | | 0.14 | | | | | 0.57 |
| Smoking Baseline | | | | | | | | | | | | |
| Never | 18 | 45.0 | 66 | 56.4 | 30 | 46.2 | | 101 | 51.3 | 13 | 52.0 | |
| Past | 19 | 47.5 | 46 | 39.3 | 32 | 49.2 | | 85 | 43.1 | 12 | 48.0 | |
| Current | 3 | 7.5 | 5 | 4.3 | 3 | 4.6 | | 11 | 5.6 | 0 | | |
| p (trend) | | | | | | | 0.95 | | | | | 0.61 |
| BMI Baseline | | | | | | | | | | | | |
| <25 | 14 | 35.0 | 41 | 35.0 | 19 | 29.2 | | 70 | 35.5 | 4 | 16.0 | |
| 25-29 | 15 | 37.5 | 51 | 43.6 | 30 | 46.2 | | 82 | 41.6 | 14 | 56.0 | |
| ≧30 | 11 | 27.5 | 25 | 21.4 | 16 | 24.6 | | 45 | 22.8 | 7 | 28.0 | |
| p (trend) | | | | | | | 0.74 | | | | | 0.12 |
| Antioxidants | | | | | | | | | | | | |
| Yes | 17 | 42.5 | 56 | 47.9 | 25 | 38.5 | | 84 | 42.6 | 14 | 56.0 | |
| No | 23 | 57.5 | 61 | 52.1 | 40 | 61.5 | | 113 | 57.4 | 11 | 44.0 | |
| p (trend) | | | | | | | 0.55 | | | | | 0.21 |

| | CFB:rs641153(R32Q) | | | | | C3:rs2230199(R102H) | | | | | | |
|---|---|---|---|---|---|---|---|---|---|---|---|---|
| | CC | | CT or TT | | | CC | | CG | | GG | | |
| | N | % | N | % | | N | % | N | % | N | % | |
| Variable | | | | | | | | | | | | |
| Baseline Age ≦70 | 119 | 70.4 | 37 | 69.8 | | 92 | 65.7 | 58 | 78.4 | 6 | 75.0 | |
| 70+ | 50 | 29.6 | 16 | 30.2 | | 48 | 34.3 | 16 | 21.6 | 2 | 25.0 | |
| p (trend) | | | | | 0.93 | | | | | | | 0.08 |
| Gender | | | | | | | | | | | | |
| Male | 76 | 45.0 | 25 | 47.2 | | 66 | 47.1 | 34 | 45.9 | 1 | 12.5 | |
| Female | 93 | 55.0 | 28 | 52.8 | | 74 | 52.9 | 40 | 54.1 | 7 | 87.5 | |
| p (trend) | | | | | 0.78 | | | | | | | 0.23 |
| Education | | | | | | | | | | | | |
| High School) | 47 | 27.8 | 17 | 32.1 | | 46 | 32.9 | 16 | 21.6 | 2 | 25.0 | |
| College+ | 122 | 72.2 | 36 | 67.9 | | 94 | 67.1 | 58 | 78.4 | 6 | 75.0 | |
| p (trend) | | | | | 0.55 | | | | | | | 0.12 |
| Smoking Baseline | | | | | | | | | | | | |
| Never | 91 | 53.8 | 23 | 43.4 | | 75 | 53.6 | 34 | 45.9 | 5 | 62.5 | |
| Past | 70 | 41.4 | 27 | 50.9 | | 58 | 41.4 | 37 | 50.0 | 2 | 52.0 | |
| Current | 8 | 4.7 | 3 | 5.7 | | 7 | 5.0 | 3 | 4.1 | 1 | 12.5 | |
| p (trend) | | | | | 0.22 | | | | | | | 0.58 |
| BMI Baseline | | | | | | | | | | | | |
| <25 | 57 | 33.7 | 17 | 32.1 | | 49 | 35.0 | 21 | 28.4 | 4 | 50.0 | |
| 25-29 | 72 | 42.6 | 24 | 45.3 | | 59 | 42.1 | 36 | 48.6 | 1 | 12.5 | |
| ≧30 | 40 | 23.7 | 12 | 22.6 | | 32 | 22.9 | 17 | 23.0 | 3 | 37.5 | |
| p (trend) | | | | | 0.96 | | | | | | | 0.64 |
| Antioxidants | | | | | | | | | | | | |
| Yes | 78 | 46.2 | 20 | 37.7 | | 59 | 42.1 | 37 | 50.0 | 2 | 25.0 | |
| No | 91 | 53.8 | 33 | 62.3 | | 81 | 57.9 | 37 | 50.0 | 6 | 75.0 | |
| p (trend) | | | | | 0.28 | | | | | | | 0.76 |

TABLE 7A

Associations Between Pairs of Genotypes Among Controls

| | |
|---|---|
| LOC387715: rs10490924(A69S) | CFH: rs1410996 |

| | | | | | |
|---|---|---|---|---|---|
| GG | GT | TT | TT | CT | CC |

| | | | | | | | | | | | | |
|---|---|---|---|---|---|---|---|---|---|---|---|---|
| Genotype | N | % | N | % | N | % | N | % | N | % | N | % |

| | | | | | | | | | | | | | | |
|---|---|---|---|---|---|---|---|---|---|---|---|---|---|---|
| CFH: | | | | | | | | | | | | | | |
| rs1061170(Y402H) | | | | | | | | | | | | | | |
| TT | 57 | 37.7 | | 32 | 47.8 | 2 | 50 | 38 | 95 | | 35 | 29.9 | 18 | 27.7 |
| CT | 72 | 47.4 | | 28 | 41.8 | 2 | 50 | 2 | 5 | | 82 | 70.1 | 18 | 27.7 |
| CC | 22 | 14.6 | | 7 | 10.4 | 0 | 0 | 0 | 0 | | 0 | 0 | 29 | 44.6 |
| p (trend) | | | 0.12 | | | | | | | <0.001 | | | | |
| LOC387715: | | | | | | | | | | | | | | |
| rs10490924(A69S) | | | | | | | | | | | | | | |
| GG | | | | | | | | 29 | 72.5 | | 76 | 65 | 46 | 70.8 |
| GT | | | | | | | | 10 | 25 | | 39 | 33.3 | 18 | 27.7 |
| TT | | | | | | | | 1 | 2.5 | | 2 | 1.7 | 1 | 1.5 |
| p (trend) | | | | | | | | | | 0.93 | | | | |
| CFH: rs1410996 | | | | | | | | | | | | | | |
| TT | | | | | | | | | | | | | | |
| CT | | | | | | | | | | | | | | |
| CC | | | | | | | | | | | | | | |
| p (trend) | | | | | | | | | | | | | | |
| C2: rs9332739(E318D) | | | | | | | | | | | | | | |
| TT | | | | | | | | | | | | | | |
| CT or CC | | | | | | | | | | | | | | |
| p (trend) | | | | | | | | | | | | | | |
| CFB: rs641153(R32Q) | | | | | | | | | | | | | | |
| CC | | | | | | | | | | | | | | |
| CT or TT | | | | | | | | | | | | | | |
| p (trend) | | | | | | | | | | | | | | |

TABLE 7B

Associations Between Pairs of Genotypes Among Controls

| | | |
|---|---|---|
| C2: rs9332739(E318D) | CFB: rs641153(R32Q) | c3: R32230199(r102h) |

| | | | | | | |
|---|---|---|---|---|---|---|
| TT | CT or CC | CC | CT or TT | CC | CG | GG |

| | | | | | | | | | | | | | | | |
|---|---|---|---|---|---|---|---|---|---|---|---|---|---|---|---|
| Genotype | p (trend) | N | % | N | % | N | % | N | % | N | % | N | % | N | % |

| | | | | | | | | | | | | | | | | | | |
|---|---|---|---|---|---|---|---|---|---|---|---|---|---|---|---|---|---|---|
| CFH: | | | | | | | | | | | | | | | | | | |
| rs1061170(Y402H) | | | | | | | | | | | | | | | | | | |
| | TT | 80 | 40.6 | | 11 | 44.0 | 68 | 40.2 | | 23 | 43.4 | 53 | 37.9 | | 33 | 44.6 | 5 | 6.8 |
| | CT | 91 | 46.2 | | 11 | 44.0 | 79 | 46.7 | | 23 | 43.4 | 72 | 51.4 | | 29 | 39.2 | 1 | 1.4 |
| | CC | 26 | 13.2 | | 3 | 12.0 | 22 | 13 | | 7 | 13.2 | 15 | 10.7 | | 12 | 16.2 | 2 | 2.7 |
| | p (trend) | | | 0.75 | | | | | 0.78 | | | | | 0.74 | | | | |
| LOC387715: | | | | | | | | | | | | | | | | | | |
| rs10490924(A69S) | | | | | | | | | | | | | | | | | | |
| | GG | 132 | 67 | | 19 | 76 | 117 | 69.2 | | 34 | 64.2 | 95 | 67.9 | | 49 | 66.2 | 7 | 87.5 |
| | CT | 62 | 31.5 | | 5 | 20 | 50 | 29.6 | | 17 | 32.1 | 43 | 30.7 | | 23 | 31.1 | 1 | 12.5 |
| | TT | 3 | 1.5 | | 1 | 4 | 2 | 1.2 | | 2 | 3.8 | 2 | 1.4 | | 2 | 2.7 | 0 | 0 |
| | p (trend) | | | 0.55 | | | | | 0.34 | | | | | 0.74 | | | | |
| CFH: | | | | | | | | | | | | | | | | | | |
| rs1410996 | | | | | | | | | | | | | | | | | | |
| | TT | 37 | 18.8 | | 3 | 12 | 29 | 17.2 | | 11 | 20.8 | 22 | 15.7 | | 16 | 21.6 | 2 | 25 |
| | CT | 101 | 51.6 | | 16 | 64 | 92 | 54.4 | | 25 | 47.2 | 82 | 58.6 | | 33 | 44.6 | 2 | 25 |
| | CC | 59 | 29.9 | | 6 | 24 | 48 | 28.4 | | 17 | 32.1 | 36 | 25.7 | | 25 | 33.8 | 4 | 50 |
| | p (trend) | | | 0.95 | | | | | 0.99 | | | | | 0.61 | | | | |
| C2: | | | | | | | | | | | | | | | | | | |
| rs9332739 | | | | | | | | | | | | | | | | | | |
| (E318D) | | | | | | | | | | | | | | | | | | |
| | TT | | | | | | 151 | 89.3 | | 46 | 86.8 | 129 | 92.1 | | 61 | 82.4 | 7 | 87.5 |
| | CT or CC | | | | | | 18 | 10.7 | | 7 | 13.2 | 11 | 7.9 | | 13 | 17.6 | 1 | 12.5 |
| | p (trend) | | | | | | | | 0.61 | | | | | 0.07 | | | | |
| CFB: | | | | | | | | | | | | | | | | | | |
| rs641153 | | | | | | | | | | | | | | | | | | |
| (R32Q) | | | | | | | | | | | | | | | | | | |
| | CC | | | | | | | | | | | 110 | 78.6 | | 57 | 77 | 2 | 25 |
| | CT or TT | | | | | | | | | | | 30 | 21.4 | | 17 | 23 | 6 | 75 |
| | | | | | | | | | | | | | | 0.03 | | | | |

TABLE 8

AMD Prevalence Rates According to Genotype.

| | | AMD | | | | |
|---|---|---|---|---|---|---|
| | | Preva- | | 95% | P | P |
| Genotype | N | lence | OR* | CI* | value | trend |
| CFH: | | | | | | |
| rs1061170 | | | | | | |
| (Y402H) | | | | | | |
| TT | 200 | 48.0 | 1.0 | | | |
| CT | 375 | 69.3 | 2.4 | (1.7-3.5) | <0.001 | |
| CC | 273 | 86.5 | 0.9 | (4.4-10.8) | <0.001 | |
| | | | | | | <0.001 |
| LOC387715: | | | | | | |
| rs10490924 | | | | | | |
| (A69S) | | | | | | |
| GG | 339 | 50.7 | 1.0 | | | |
| GT | 346 | 77.5 | 3.4 | (2.4-4.6) | <0.001 | |
| TT | 117 | 94.9 | 18.0 | (7.7-41.9) | <0.001 | |
| | | | | | <0.001 | |
| CFH: | | | | | | |
| rs1410996 | | | | | | |
| TT | 66 | 31.8 | 1.0 | | | |
| CT | 288 | 56.9 | 2.8 | (1.6-5) | <0.001 | |
| CC | 451 | 83.8 | 11.1 | (6.2-19.7) | <0.001 | |
| | | | | | | <0.001 |
| C2: | | | | | | |
| rs9332739 | | | | | | |
| (E318D) | | | | | | |
| TT | 780 | 71.5 | 1.0 | | | |
| CT or CC | 53 | 49.1 | 0.4 | (0.2-0.7) | <0.001 | |
| CFB: | | | | | | |
| rs641153 | | | | | | |
| (R32Q) | | | | | | |
| CC | 722 | 74.0 | 1.0 | | | |
| CT or TT | 113 | 44.3 | 0.3 | (0.2-0.4) | <0.001 | |
| C3: | | | | | | |
| rs223019 | | | | | | |
| 9(R102H) | | | | | | |
| CC | 451 | 63.4 | 1.0 | | | |
| CG | 339 | 74.6 | 1.7 | (1.2-2.3) | <0.001 | |
| GG | 58 | 84.5 | 3.1 | (1.5-6.6) | 0.002 | |
| | | | | | | <0.001 |

*OR = odds ratio; CI = confidence interval

TABLE 9
Association Between Advanced AMD and Demographic, Behavioral and Genetic Risk Factors.

| | All Advanced | | Unilateral advanced | | Bilateral advanced | | Geographic | | Neovascular | |
|---|---|---|---|---|---|---|---|---|---|---|
| | AMD | | AMD† | | AMD† | | atrophy‡ | | AMD‡ | |
| | OR | p- | OR | p- | OR | p- | OR | p- | OR | p- |
| | (95% CI)* | value | (95% CI) | value | (95% CI) | value | (95% CI) | value | (95% CI) | value |
| # Cases/Controls | 509/222 | | 202/222 | | 307/222 | | 136/222 | | 373/222 | |
| Variable | | | | | | | | | | |
| Age (yr) <70 | 1.0 | | 1.0 | | 1.0 | | 1.0 | | 1.0 | |
| ≧70 | 2.8 | | 2.3 | | 3.7 | | 2.6 | | 3.1 | |
| | (1.8-4.2) | <0.0001 | (1.4-3.8) | 0.001 | (2.2-6.2) | <0.0001 | (1.5-4.6) | 0.001 | (1.9-4.9) | <0.0001 |
| Gender | | | | | | | | | | |
| Female | 1.0 | | 1.0 | | 1.0 | | 1.0 | | 1.0 | |
| Male | 0.9 | | 1.0 | | 0.9 | | 1.0 | | 0.9 | |
| | (0.6-1.4) | 0.62 | (0.6-1.5) | 0.85 | (0.5-1.4) | 0.55 | (0.6-1.8) | 0.89 | (0.5-1.3) | 0.5 |
| Education | | | | | | | | | | |
| ≦High School | 1.0 | | 1.0 | | 1.0 | | 1.0 | | 1.0 | |
| >High School | 0.6 | | 0.5 | | 0.6 | | 0.7 | | 0.6 | |
| | (0.4-0.9) | 0.01 | (0.3-0.9) | 0.01 | (0.4-1.0) | 0.07 | (0.4-1.2) | 0.18 | (0.3-0.9) | 0.01 |
| Smoking | | | | | | | | | | |
| Never | 1.0 | | 1.0 | | 1.0 | | 1.0 | | 1.0 | |
| Past | 1.9 | | 2.2 | | 1.6 | | 1.8 | | 1.9 | |
| | (1.2-2.9) | 0.004 | (1.3-3.6) | 0.002 | (0.9-2.6) | 0.09 | (1.0-3.1) | 0.06 | (1.2-3.1) | 0.01 |
| Current | 3.9 | | 3.7 | | 4.0 | | 2.7 | | 4.4 | |
| | (1.7-8.9) | 0.001 | (1.5-9.6) | 0.01 | (1.5-10.7) | 0.01 | (0.8-8.9) | 0.11 | (1.9-10.4) | 0.001 |
| BMI | | | | | | | | | | |
| <25 | 1.0 | | 1.0 | | 1.0 | | 1.0 | | 1.0 | |
| 25-29 | 1.1 | | 1.2 | | 1.0 | | 1.0 | | 1.1 | 0.65 |
| | (0.7-1.8) | 0.72 | (0.7-2.1) | 0.53 | (0.6-1.8) | 0.99 | (0.5-1.9) | 0.97 | (0.7-1.9) | |
| 30+ | 1.5 | | 1.7 | | 1.5 | | 2.7 | | 1.8 | |
| | (0.9-2.6) | 0.11 | (0.9-3.2) | 0.09 | (0.8-2.9) | 0.25 | (0.8-8.9) | 0.44 | (1.0-3.2) | 0.06 |
| Antioxidant | | | | | | | | | | |
| No | 1.0 | | 1.0 | | 1.0 | | 1.0 | | 1.0 | |
| Yes | 1.3 | | 1.3 | | 1.2 | | 1.1 | | 1.4 | |
| | (0.8-1.9) | 0.25 | (0.8-2.1) | 0.29 | (0.7-2.0) | 0.42 | (0.6-1.9) | 0.77 | (0.9-2.2) | 0.14 |

TABLE 10
Interaction Effects of BMI, Smoking, and Genotype on Risk of Advanced AMD.

| | | | | |
|---|---|---|---|---|
| BMI | P | | P | |
| OR (95% CI)* | (inter- | P | inter- | P |

| | | | | | | | | |
|---|---|---|---|---|---|---|---|---|
| Variable | <25 | 25+ | action) | trend | Never | Ever | action | Trend |

| | | | | | | | | |
|---|---|---|---|---|---|---|---|---|
| CFH: | | | | | | | | |
| rs1061170 | | | | | | | | |
| (Y402H) | | | | | | | | |
| TT | 1.0 | 0.6 | | | 1.0 | 1.6 | | |
| | | (0.3-1.4) | | | | (0.8-3.4) | | |
| CT | 0.9 | 1.6 | 0.035 | | 1.3 | 3.6 | 0.26 | |
| | (0.4-2.0) | (0.8-3.3) | (CT vs TT) | | (0.6-2.7) | (1.8-7.4) | (CT vs TT) | |
| CC | 1.8 | 2.8 | 0.14 | | 3.5 | 5.1 | 0.85 | |
| | (0.6-5.2) | (1.1-6.9) | (CC vs TT) | | (1.3-9.1) | (2.1-12.3) | (CC vs TT) | |
| | | | | 0.090 | | | | 0.97 |
| LOC387715: | | | | | | | | |
| rs10490924 | | | | | | | | |
| (A69S) | | | | | | | | |
| GG | 1.0 | 1.3 | | | 1.0 | 2.5 | | |
| | | (0.7-2.3) | | | | (1.4-4.3) | | |
| GT | 3.3 | 3.9 | 0.81 | | 4.2 | 6.0 | 0.20 | |
| | (1.6-6.9) | (2.1-7.2) | (GT vs GG) | | (2.2-7.8) | (3.4-10.8) | (GT vs GG) | |
| TT | 25.9 | 32.1 | 0.96 | | 17.4 | 120.4 | 0.40 | |
| | (3.2-211.1) | (8.7-118.3) | (TT vs GG) | | (4.7-63.5) | (15.1-957.2) | (TT vs GG) | |
| | | | | 0.90 | | | | 0.57 |
| CFH: | | | | | | | | |
| rs1410996 | | | | | | | | |
| TT | 1.0 | 2.0 | | | 1.0 | 2.1 | | |
| | | (0.5-8.0) | | | | (0.6-7.9) | | |
| CT | 2.4 | 2.8 | 0.46 | | 1.4 | 4.0 | 0.70 | |
| | (0.7-8.4) | (0.8-9.6) | (CT vs TT) | | (0.4-4.5) | (1.3-12.7) | (CT vs TT) | |
| CC | 5.3 | 6.4 | 0.50 | | 4.6 | 6.5 | 0.58 | |
| | (1.4-20.2) | (1.8-22.7) | (CC vs TT) | | (1.4-15.2) | (2.0-21.6) | (CC vs TT) | |
| | | | | 0.65 | | | | 0.22 |
| C2: | | | | | | | | |
| rs9332739 | | | | | | | | |
| (E318D) | | | | | | | | |
| TT | 1.0 | 1.3 | | | 1.0 | 1.9 | | |
| | | (0.8-2.0) | | | | (1.3-3.0) | | |
| CT or CC | 0.6 | 0.3 | 0.44 | | 0.2 | 0.8 | 0.34 | |
| | (0.1-3.9) | (0.1-0.6) | (CT-CC vs TT) | | (0.05-0.7) | (0.3-2.2) | (CT-CC vs TT) | |
| CFB: | | | | | | | | |
| rs641153 | | | | | | | | |
| (R32) | | | | | | | | |
| CC | 1.0 | 1.3 | | | 1.0 | 2.1 | | |
| | | (0.8-2.0) | | | | (1.3-3.2) | | |
| CT or TT | 0.3 | 0.3 | 0.9 | | 0.3 | 0.5 | 0.82 | |
| | (0.1-0.7) | (0.1-0.6) | (CT-TT vs CC) | | (0.1-0.6) | (0.2-1.0) | (CT-TT vs CC) | |
| C3: | | | | | | | | |
| rs2230199 | | | | | | | | |
| (R102H) | | | | | | | | |
| CC | 1.0 | 1.5 | | | 1.0 | 2.2 | | |
| | | (0.9-2.7) | | | | (1.3-3.8) | | |
| CG | 2.4 | 2.1 | 0.21 | | 1.9 | 3.3 | 0.54 | |
| | (1.2-5.1) | (1.1-3.9) | (CG vs CC) | | (1.0-3.6) | (1.8-5.9) | (CG vs CC) | |
| GG | 2.5 | 7.2 | 0.51 | | 3.3 | 9.8 | 0.77 | |
| | (0.5-11.1) | (1.9-27.2) | (GG vs CC) | | (1.0-10.9) | (2.0-47.5) | (GG vs CC) | |
| | | | | 0.62 | | | | 0.73 |

*OR = Odds Ratio,
CI = confidence interval
OR's adjusted for age (<70, ≧70), gender, education (≦high school, >high school), smoking (never, past, current), BMI (25, 25-29, 30+),
antioxidant treatment (yes, no), and all genetic variants and associated genotypes.

TABLE 11A

Assessment of Gene-Gene Interactions Associated
with Advanced Age-Related Macular
Degeneration*.
Genetic LOC387715: rs10490924
(A69S)

| | | | | p- |
|---|---|---|---|---|
| Variant | GG | GT | TT | value |
| CFH: | | | | |
| rs1061170 | | | | |
| (Y402H) | | | | |
| TT | 1.0 | 2.7 | 12.9 | 0.28 |
| | | (1.3-5.7) | (2.3-71.4) | |
| CT | 1.6 | 4.5 | 45.1 | |
| | (0.8-3.3) | (2.1-9.4) | (9.5-215.2) | |
| CC | 2.5 | 12.1 | † | |
| | (1.1-6.0) | (4.3-34.3) | | |
| L0C387715: | | | | |
| rs10490924 | | | | |
| (A69S) | | | | |
| GG | | | | |
| GT | | | | |
| TT | | | | |
| CFH: | | | | |
| rs1410996 | | | | |
| TT | | | | |
| CT | | | | |
| CC | | | | |
| C2: | | | | |
| rs9332739 | | | | |
| (E318D) | | | | |
| TT | | | | |
| CT or CC | | | | |
| CFB: | | | | |
| rs641153 | | | | |
| (R32Q) | | | | |
| CC | | | | |
| CT or TT | | | | |

*OR's adjusted for age (<70, ≧70), gender, education (≦high school, >high school), smoking (never, past, current), BMI (25, 25-29, 30+),
antioxidant treatment (yes, no), and all genetic variants and associated genotypes as listed in table.
† No controls in this category
‡ No Cases in this category

TABLE 11 B

Assessment of Gene-Gene Interactions Associated
with Advanced Age-Related Macular
Degeneration*.
Genetic Variant CFH: rs1410996

| | | | |
|---|---|---|---|
| | | | p- |
| | | | val- |
| TT | CT | CC | ue |

| | | | | |
|---|---|---|---|---|
| CFH: | | | | |
| rs106117 | | | | |
| 0 | | | | |
| (Y402H) | | | | |
| TT | 1.0 | 2.7 | 1.6 | 0.05 |
| | | (1.2-6.3) | (0.6-4.3) | |
| CT | 3.0 | 2.3 | 10.7 | |
| | (0.3-32.3)† | (1.1-4.9) | (4.5-25.4) | |
| CC | 12.5 | † | 12.5 | |
| | (5.7-27.3)†† | | (5.7-27.3) | |
| LOC3877 | | | | |
| 15: | | | | |
| rs104909 | | | | |
| 24 | | | | |
| (A69S) | | | | |
| GG | 1.0 | 2.2 | 2.9 | 0.07 |
| | | (0.8-5.8) | (1.0-8.1) | |
| GT | 3.8 | 3.7 | 16.7 | |
| | (1.0-13.9) | (1.4-9.9) | (5.9-47.6) | |
| TT | 4.2 | 51.4 | 126.1 | |
| | (0.2-100.8) | (9.3-283.8) | (13.8-1154.1) | |
| CFH: | | | | |
| rs141099 | | | | |
| 6 | | | | |
| TT | | | | |
| CT | | | | |
| CC | | | | |
| C2: | | | | |
| rs933273 | | | | |
| 9 | | | | |
| (E318D) | | | | |
| TT | | | | |
| CT or CC | | | | |
| CFB: | | | | |
| rs641153 | | | | |
| (R32Q) | | | | |
| CC | | | | |
| CT or TT | | | | |

TABLE 11 C

Assessment of Gene-Gene
Interactions Associated with Advanced Age-
Related Macular Degeneration*.
C2: rs9332739(E318D)

| | | | |
|---|---|---|---|
| Genetic Variant | TT | CT or CC | p-value |

| | | | |
|---|---|---|---|
| CFH: | | | |
| rs1061170 | | | |
| (Y402H) | | | |
| TT | 1.0 | 0.3(0.1-1.0) | 0.60 |
| CT | 1.7(1.0-2.9) | 0.4(0.1-1.5) | |
| CC | 3.3(1.6-6.6) | 1.3(0.3-5.8) | |
| LOC387715: | | | |
| rs10490924 | | | |
| (A69S) | | | |
| GG | 1.0 | 0.3(0.1-0.8) | 0.77 |
| GT | 3.0(1.9-4.5) | 1.4(0.4-5.0) | |
| TT | 28.1(8.3-95.5) | 3.8(0.4-39.6) | |
| CFH: | | | |
| rs1410996 | | | |
| TT | 1.0 | 0.6(0.1-6.3) | 0.75 |
| CT | 1.7(0.8-3.8) | 0.3(0.1-1.4) | |
| CC | 3.8(1.6-8.7) | 1.5(0.4-5.6) | |
| C2: | | | |
| rs9332739 | | | |
| (E318D) | | | |
| TT | | | |
| CT or CC | | | |
| CFB: | | | |
| rs641153 | | | |
| (R32Q) | | | |
| CC | | | |
| CT or TT | | | |

TABLE 11 D

Assessment of Gene-Gene
Interactions Associated with Advanced
Age-Related Macular Degeneration*.
CFB: rs641153(R32Q)

| | | | p- |
|---|---|---|---|
| Genetic Variant | CC | CT or TT | value |
| CFH: rs1061170 | | | |
| (Y402H) | | | |
| TT | 1.0 | 0.2(0.1-0.6) | 0.89 |
| CT | 1.6(1.0-2.8) | 0.5(0.2-1.1) | |
| CC | 3.4(1.6-7.0) | 0.6(0.2-2.1) | |
| LOC387715: | | | |
| rs10490924 | | | |
| (A69S) | | | |
| GG | 1.0 | 0.3(0.1-0.6) | 0.54 |
| GT | 3.2(2.0-5.0) | 0.7(0.3-1.7) | |
| TT | 37.0(8.5-160.7) | 3.1(0.6-16.1) | |
| CFH: rs1410996 | | | |
| TT | 1.0 | 0.1(0.0-0.9) | 0.74 |
| CT | 1.4(0.6-3.2) | 0.5(0.2-1.5) | |
| CC | 3.5(1.5-8.4) | 0.7(0.2-2.0) | |
| C2: rs9332739 | | | |
| (E318D) | | | |
| TT | 1.0 | 0.3(0.1-0.5) | 0.70 |
| CT or CC | 0.3(0.1-0.8) | 0.1(0.0-0.4) | |
| CFB: rs641153 | | | |
| (R32Q) | | | |
| CC | | | |
| CT or TT | | | |

TABLE 11 E

Assessment of Gene-Gene Interactions
Associated with Advanced Age-Related Macular
Degeneration*.
Genetic Variant C3: rs2230199(R102H

| | | | |
|---|---|---|---|
| | | | p- |
| | | | val- |
| CC | CG | GG | ue |

| | | | | |
|---|---|---|---|---|
| CFH: | | | | |
| rs1061170 | | | | |
| (Y402H) | | | | |
| TT | 1.0 | 1.3 | 0.9 | 0.67 |
| | | (0.6-2.7) | (0.1-6.5) | |
| CT | 1.3 | 2.7 | 20.7 | |
| | (0.7-2.4) | (1.3-5.6) | (2.5-174.4) | |
| CC | 3.2 | 4.1 | 5.3 | |
| | (1.3-7.6) | (1.6-10.3) | (0.8-33.8) | |
| LOC387715: | | | | |
| rs10490924 | | | | |
| (A69S) | | | | |
| GG | 1.0 | 1.2 | 2.3 | 0.12 |
| | | (0.7-2.1) | (0.7-7.1) | |
| GT | 2.2 | 5.6 | 22.4 | |
| | (1.3-3.7) | (3.0-10.5) | (2.5-198.9) | |
| TT | 22.9 | 31.3 | † | |
| CFH: | (5.2-101.8) | (6.5-150.3) | | |
| rs1410996 | | | | |
| TT | 1.0 | 0.7 | ‡ | 0.91 |
| | | (0.2-2.8) | | |
| CT | 1.0 | 2.1 | 9.5 | |
| | (0.4-2.6) | (0.8-5.6) | (1.5-58.9) | |
| CC | 2.8 | 4.2 | 6.0 | |
| | (1.0-7.4) | (1.5-11.6) | (1.3-27.6) | |
| C2: | | | | |
| rs9332739 | | | | |
| (E318D) | | | | |
| TT | 1.0 | 1.8 | 4.0 | 0.06 |
| | | (1.2-2.8) | (1.5-10.7) | |
| CT or CC | 0.7 | 0.3 | 0.1 | |
| | (0.2-2.0) | (0.1-0.9) | (0-182.4) | |
| CFB: | | | | |
| rs641153 | | | | |
| (R32Q) | | | | |
| CC | 1.0 | 1.7 | 8.6 | 0.11 |
| | | (1.1-2.7) | (1.9-40.0) | |
| CT or TT | 0.3 | 0.4 | 0.3 | |
| | (0.2-0.7) | (0.2-0.9) | (0.1-1.5) | |

TABLE 12
C Statistics for Advanced AMD Based on Models with Different
Combinations of Genetic and Environmental Variables.

| | | | Demographic, | C |
|---|---|---|---|---|
| | | Genetic | Environmental | Statistic |
| Model | Sample | Variables | Variables | (+/−SE)* |
| 1 | original | CFH Y402H, | Age, gender, | 0.803 +/− |
| | | LOC387715 A69S | education, | 0.018 |
| | | | antioxidant | |
| | | | treatment | |
| 2 | original | CFH Y402H, | Age, gender, | 0.822 +/− |
| | | LOC387715 A69S | education, | 0.017 |
| | | | antioxidant | |
| | | | treatment, | |
| | | | smoking | |
| | | | BMI | |
| 3 | original | CFH Y402H, | Age, gender, | 0.846 +/− |
| | | LOC387715 A69S, | education, | 0.016 |
| | | CFH 1410996, | antioxidant | |
| | | C2E318D, | treatment | |
| | | CFB R32Q, C3 | | |
| | | R102H | | |
| 4 | original | CFH Y402H, | Age, gender, | 0.859 +/− |
| | | LOC387715 A69S, | education, | 0.015 |
| | | CFH 1410996, | antioxidant | |
| | | C2E318D, | treatment, | |
| | | CFB R32Q, C3 | smoking | |
| | | R102H | BMI | |
| 4a | replication | CFH Y402H, | Age, gender, | 0.810 +/− |
| | | LOC387715 A69S, | education, | 0.016 |
| | | CFH 1410996, | antioxidant | |
| | | C2E318D, | treatment, | |
| | | CFB R32Q, C3 | smoking | |
| | | R102H | BMI | |

*p value (model 1 vs 2, p = 0.027; 1 vs 3 p < 0.001; 2 vs 4, p = 0.001, 3 vs 4, p = 0.037)

TABLE 13

Baseline Demographic and Genetic Characteristics of Participants

| | | |
|---|---|---|
| Family | Twin | Total |
| N = 1620 | N = 506 | N = 2126 |

| | | | |
|---|---|---|---|
| Mean Age | 76.6 +/− 9.4 | 77.8 +/− 5.1 | 76.9 +/− 8.6 |
| (+/− SD) | | | |
| Gender | | | |
| M (%) | 665 (41%) | 100% | 1170 (55%) |
| F (%) | 955 (59%) | | 955 (45%) |
| Genotype | | | |

CFH Y402H: rs1061170

| | | | | |
|---|---|---|---|---|
| TT | | 330 (21) | 157 (32) | 487 (24) |
| CT | | 733 (47) | 221 (45) | 954 (46) |
| CC | risk allele is C | 508 (32) | 112 (23) | 620 (30) |

CFH: rs1410996

| | | | | |
|---|---|---|---|---|
| TT | | 114 (7) | 64 (13) | 174 (9) |
| CT | | 569 (36) | 196 (42) | 765 (38) |
| CC | risk allele is C | 880 (56) | 213 (45) | 1093 (54) |

LOC387715: rs10490924(A69S)

| | | | | |
|---|---|---|---|---|
| GG | | 591 (38) | 263 (54) | 854 (42) |
| GT | | 675 (44) | 182 (38) | 857 (42) |
| TT | risk allele is T | 280 (18) | 40 (8) | 320 (16) |

C2: rs9332739(E318D)

| | | | | |
|---|---|---|---|---|
| GG | | 1447 (93) | 449 (93) | 1896 (93) |
| CG/CC | protective allele | 117 (7) | 35 (7) | 152 (7) |
| | is C | | | |

CFB: rs641153(R32Q)

| | | | | |
|---|---|---|---|---|
| CC | | 1351 (87) | 429 (88) | 1780 (87) |
| CT/TT | protective allele | 204 (13) | 57 (12) | 261 (13) |
| | is T | | | |

C3: rs2230199 (R102G)

| | | | | |
|---|---|---|---|---|
| CC | | 712 (49) | 218 (48) | 930 (49) |
| CG | | 631 (43) | 190 (42) | 821 (43) |
| GG | risk allele is G | 115 (8) | 45 (10) | 160 (8) |

### REFERENCES

All references cited herein and throughout this specification are hereby incorporated herein by reference in their entirety.
Abecasas, G. et. al., Nat. Genet. 38 (9): 1049-1054 (2006).
Abrera-Abeleda M, et al., J. Med. Genet. 43: 582-589 (2006).
Age-Related Eye Disease Study Research group. AREDS Report No. 8. Arch Opthalmol., 119:1417-1436 (2001).
Age-Related Eye Disease Study Research Group. AREDS Report No. 22. Arch Opthalmol., 125:1225-1232 (2007).
Age-Related Eye Disease study Group. AREDS Report No. 20. Arch Opthalmol., 125:671-9 (2007).
Barrett, J. et. al., Bioinformatics 21:263-5 (2005).
Botto, M. et. al., J. Exp. Med., 172:1011-7 (1990).
Brown, K. et. al., New Eng. J. Med., 354:2014-2123 (2006).
Catterall, C. et. al., Biochem. J., 242: 849-56 (1987).
D'Agostino, R. et. al., JAMA, 286:180-187 (2001).
Dewan, A. et. al., Science, 314:989-992 (2006).
Diabetes Genetics Initiative of Broad Institute of Harvard and MIT, Lund University, and Novartis Institutes of BioMedical Research et al., Science, 316:1331-1336 (2007).
Easton, D. et. al., Nature, 447:1087-93 (2007).
Edwards, A. et al., Science, 308:421-424 (2005).
Fisher, S. et. al., Hum. Mol. Genet., 14:2257-64 (2005).
Francis, P. et. al., Hum Hered., 63:212-218 (2007).
Gold, B. et al. Nat. Genet., 38:458-462 (2006).
Haddad, S. et. al., Surv. Opthalmol. 51:316-363 (2006).
Hageman, G. et al., Proc. Natl. Acad. Sci. USA, 102:7227-7232 (2005).
Haines, J. et al., Science, 308:419-421 (2005).
Hanley J and McNeil B. Radiology, 143:29-36 (1982).
Huhtinen, H. et. al. APMIS, 114:127-30 (2006).
Ho, I. et. al., J. Biol. Chem., 276:8321-6 (2001).
Jakobsdottir, J. et. al., Am. J. Hum. Gen., 77:389-407 (2005).
Jha, P. et. al., Mol. Immunol.; 44:3901-3908 (2007).
Kanda, A. et. al. PNAS, 104:16227-16232 (2007).
Klein, R. et al., Science, 308:385-389 (2005).
Landreville, S. et. al., Invest. Opthalmol. Vis. Sci., 45:3997-4003 (2004).
Li, M. et. al., Nat. Genet., 38:1049-1054 (2006).
Maller, J. et al., Nat. Genet., 38:1055-1059 (2006).
Maller, J. et. al., Nat. Gen., 39:1200-1201 (2007).
Mares-Perlman, J A. et. al., Am. J. Epidem., 153: 424-32 (2001).
National Eye Institute (NEI) Age-Related Eye Disease Study (AREDS) (at the world wide web site: ncbi.nlm.nih.gov/projects/gap/cgi-bin/study.cgi?id=phs000001).
Nozaki, M. et. al., Proc. Natl. Acad. Sci. USA, 103:2328-33 (2006).
Nussenblatt, R. and Ferris, F., Am. J. Opthalmol., 144:618-626 (2007).
Poznansky, M. et. al., J. Immunol., 143:1254-1258 (1989).
Purcell, S. et al., Am. J. Hum. Genet., 81:559-75 (2007).
Rivera A. et al., Hum. Mol. Genet., 14:3227-36 (2005).
Rosner B. Fundamentals of Biostatistics. 6^{th} Edition. Boston: Duxbury Press (2005).
Samani, N. et. al., N. Engl. J. Med., 357:443-53 (2007).
Schaumberg, D. et. al., Arch Opthalmol., 125:55-62 (2007).
Seddon, J. et. al., JAMA., 272:1413-1420 (1994).
Seddon, J. et. al., JAMA., 276:1141-1146 (1996).
Seddon, J. et. al., Am. J. Opthalmol., 134:842-8 (2002).
Seddon, J. et. al., Am. J. Hum. Gen., 73:780-790 (2003).
Seddon, J. et. al., Arch Opthalmol., 121:785-792 (2003).
Seddon, J. et. al., Arch Opthalmol., 121:1728-1737 (2003).
Seddon, J. et. al., Arch Opthalmol. 123:321-327 (2005).
Seddon, J. et. al., Hum Hered., 61:157-165 (2006).
Seddon, J. et. al., Opthalmol., 113:260-6 (2006).
Seddon, J. et. al., Arch Opthalmol., 124:995-1001 (2006).
Seddon J, Sobrin L. Epidemiology of age-related macular degeneration AMD. In Albert D M, Miller J, (eds). The Principles and Practice of Opthalmology, 3rd edition, Philadelphia: W.B. Saunders; (2007).
Seddon, J. et. al., JAMA, 297:1793-1800 (2007).
Seddon J, Sobrin L: Epidemiology of age-related macular degeneration. In Albert D M, Miller J, (eds): The Principles and Practice of Opthalmology, 3rd edition.
Philadelphia 2007: W.B. Saunders. pp. 413-422.
Snow, K. et al., Am. J. Opthalmol, 134:842-848 (2002).
Sullivan P F, Purcell S: Analyzing genome-wide association study data: a tutorial using PLINK; in Neale B M, Ferreira M A R, Medland S E, & Posthuma D (eds): Statistical Genetics: Gene Mapping Through Linkage and Association. Taylor and Table Francis, London, 2007, pp. 355-394.
Tomany, S. et. al. Opthalmology, 111:1280-1287 (2004).
Vyse, T. et. al., Genomics, 24:90-8 (1994).
Walport M., N. Engl. J. Med., 344:1140-1144 (2001).
Wiltshire, S. et. al., Eur. J. Hum. Genet., 14:1209-1214 (2006).
Wyatt, R. et. al., Plenum Press, New Sissons, 1569-1575 (1987).
Yang, Z. et. al., Science, 314:992-993 (2006).
Yates, J. et. al., NEJM, 357:553-61 (2007).
Zipfel, P. et. al., Mol. Immun., 43:97-106 (2006).

What is claimed is:

1. A method for diagnosing AMD or a susceptibility to AMD, a protective phenotype for AMD, or a neutral genotype for AMD, comprising detecting the presence or absence of a particular allele at a polymorphic site associated with a complement pathway gene, wherein the allele is indicative of AMD or a susceptibility to AMD.

2. The method of claim 1, wherein the polymorphic site is a single nucleotide polymorphism associated with complement factor 3.

3. The method of claim 1, wherein the polymorphic site is rs2230199 (SEQ ID NO:1), wherein the guanine allele is indicative of AMD or susceptibility to AMD.

4. The method of claim 3, wherein the cytosine allele is detected by detecting a C3 polypeptide comprising a glycine at amino acid position 102.

5. The method of claim 1, wherein the polymorphic site is selected from the group consisting of: rs1061170 (SEQ ID NO:2), wherein the cytidine allele is indicative of AMD or susceptibility to AMD; rs10490924 (SEQ ID NO:3), wherein the thymine allele is indicative of AMD or susceptibility to AMD; rs9332739 (SEQ ID NO:4), wherein the cytidine allele confers a protective effect against AMD; rs641153 (SEQ ID NO: 5), wherein the thymine allele confers a protective effect against AMD; rs1410996 (SEQ ID NO:6), wherein the cytidine allele is indicative of AMD or susceptibility to AMD; and rs2230203 (SEQ ID NO:7), wherein the cytidine allele is indicative of AMD or susceptibility to AMD.

6. The method of claim 1, wherein the presence or absence of a particular allele is detected by a hybridization assay.

7. The method of claim 1, wherein the presence or absence of a particular allele is determined using a microarray.

8. The method of claim 1, wherein the presence or absence of a particular allele is determined using an antibody.

9. A method for identifying a subject who is at risk or protected from developing AMD, comprising:
a) detecting the presence or absence of at least one at risk allele at rs2230199;
b) detecting the presence or absence of at least one at risk allele or protective associated with complement factor H;
c) detecting the presence or absence of at least one at risk allele or protective allele associated at LOC387715 in HTRA1; and
d) detecting the presence or absence of at least one at risk allele or protective allele associated with complement factor B,
wherein a subject is not at risk if the subject is one of about 20% of the population with a less than about 1% risk of developing AMD, and the subject is at risk if the subject is one of about 1% of the population with a greater than about 50% risk of developing AMD.

10. The method of claim 1, wherein the presence or absence of a particular allele is detected by a hybridization assay.

11. The method of claim 1, wherein the presence or absence of a particular allele is determined using a microarray.

12. A purified polynucleotide comprising the polymorphic site and at least about six or more contiguous nucleotides of one or more of the sequences given as SEQ ID NOS:1-7, wherein the variant allele is present at the polymorphic site.

13. A diagnostic array comprising one or more polynucleotide probes that are complementary to a polynucleotide of claim 12.

14. A diagnostic system comprising: a diagnostic array of claim 13, an array reader, an image processor, a database having data records and information records, a processor, and an information output; wherein the system compiles and processes patient data and outputs information relating to the statistical probability of the patient developing AMD.

15. A method of using the diagnostic system of claim 14, comprising contacting a subject sample to the diagnostic array under high stringency hybridization conditions; inputting patient information into the system; and obtaining from the system information relating to the statistical probability of the patient developing AMD.

16. A method of making the diagnostic array of claim 13, comprising: applying to a substrate at a plurality particular address on the substrate a sample of the individual purified polynucleotide compositions comprising SEQ ID NOS:1-7.

17. A method for diagnosing AMD or a susceptibility to AMD in a subject comprising combining genetic risk with behavioral risk, wherein the genetic risk is determined by detecting the presence or absence of a particular allele at a polymorphic site associated with a complement pathway gene, wherein the allele is indicative of AMD or a susceptibility to AMD.

18. The method of claim 17, wherein the polymorphic site is rs2230199 (SEQ ID NO:1), wherein the guanine allele is indicative of AMD or susceptibility to AMD.

19. The method of claim 18, wherein the cytosine allele is detected by detecting a C3 polypeptide comprising a glycine at amino acid position 102.

20. The method of claim 17, wherein the polymorphic site is selected from the group consisting of: rs1061170 (SEQ ID NO:2), wherein the cytidine allele is indicative of AMD or susceptibility to AMD; rs10490924 (SEQ ID NO:3), wherein the thymine allele is indicative of AMD or susceptibility to AMD; rs9332739 (SEQ ID NO:4), wherein the cytidine allele confers a protective effect against AMD; rs641153 (SEQ ID NO:5), wherein the thymine allele confers a protective effect against AMD; rs1410996 (SEQ ID NO:6), wherein the cytidine allele is indicative of AMD or susceptibility to AMD; and rs2230203 (SEQ ID NO:7), wherein the cytidine allele is indicative of AMD or susceptibility to AMD.

21. The method of claim 1, wherein the presence or absence of a particular allele is detected by a hybridization assay.

22. The method of claim 1, wherein the presence or absence of a particular allele is determined using a microarray.

23. The method of claim 1, wherein the presence or absence of a particular allele is determined using an antibody.

24. The method of claim 17, wherein behavioral risk is assessed by determining if the subject exhibits a behavior or trait selected from the group consisting of: obesity, smoking, vitamin and dietary supplement intake, use of alcohol or drugs, poor diet and a sedentary lifestyle.

25. The method of claim 24, wherein elevated BMI is used to determine obesity.
